(19) United States
(12) Patent Application Publication
(10) Pub. No.: US 2022/0388972 A1
(43) Pub. Date: Dec. 8, 2022

(54) CHROMAN-4-ONE DERIVATIVES FOR THE TREATMENT AND PROPHYLAXIS OF HEPATITIS B VIRUS INFECTION

(71) Applicant: Hoffmann-La Roche Inc., Little Falls, NJ (US)

(72) Inventors: Dongdong CHEN, Shanghai (CN); Song FENG, Shanghai (CN); Yongfu LIU, Shanghai (CN); Xuefei TAN, Shanghai (CN); Jiamin ZHENG, Shanghai (CN)

(73) Assignee: Hoffmann-La Roche Inc., Little Falls, NJ (US)

(21) Appl. No.: 17/413,416

(22) PCT Filed: Dec. 12, 2019
(86) PCT No.: PCT/EP2019/084772

§ 371 Date: Jun. 11, 2021

(30) Foreign Application Priority Data
Dec. 14, 2018 (CN) PCT/CN2018/121175

(51) Int. Cl. C07D 311/28; A61P 31/12

(52) U.S. Cl. CPC C07D 311/28; A61P 31/12

(57) ABSTRACT

The present invention provides compounds having the general formula (I) wherein R¹ to R¹⁰, Gi, G₂, X and m are as described herein, compositions including the compounds and methods of using the compounds for treating hepatitis B.

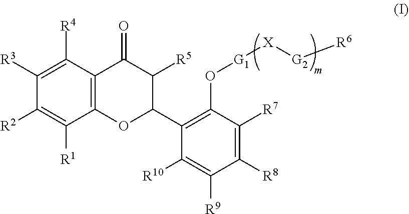

The present invention relates to organic compounds useful for therapy and/or prophylaxis of HBV infection in a mammal, and in particular to cccDNA (covalently closed circular DNA) inhibitors useful for treating HBV infection.

### FIELD OF THE INVENTION

The present invention relates to chroman-4-one derivatives having pharmaceutical activity, their manufacture, pharmaceutical compositions containing them and their potential use as medicaments.

The present invention relates to compounds of formula (I)

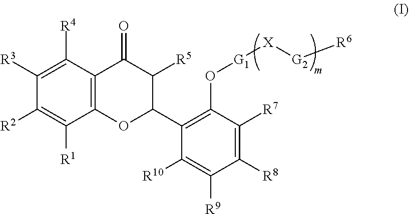

wherein R¹ to R¹⁰, G₁, G₂, X and m are as described below, or a pharmaceutically acceptable salt thereof.

Hepatitis B virus (HBV) infection is one of the most prevalent viral infections and is a leading cause of chronic hepatitis. It is estimated that worldwide, around 2 billion people have evidence of past or present infection with HBV. Over 250 million individuals are currently chronically infected with HBV and are therefore at high risk to develop liver fibrosis, cirrhosis and hepatocellular carcinoma (HCC). There are data to indicate ˜800,000 deaths per year are directly linked to HBV infection (Lozano, R. et al., Lancet (2012), 380 (9859), 2095-2128; Goldstein, S. T. et al., Int J Epidemiol (2005), 34 (6), 1329-1339).

Many countries in the world administer hepatitis B vaccine starting at birth or in early childhood, which has greatly reduced the incidence and prevalence of hepatitis B in most endemic regions over the past few decades. However, the vaccine has no impact on people who were infected before the widely use of the vaccine in developing end-stage liver disease or HCC (Chen, D. S., J Hepatol (2009), 50 (4), 805-816). Vaccination at birth of infants born to HBV positive mothers is usually not sufficient for protecting vertical transmission and combination with hepatitis B immune globulin is needed (Li, X. M. et al., World J Gastroenterol (2003), 9 (7), 1501-1503).

Currently FDA-approved treatments for chronic hepatitis B include two type 1 interferons (IFN) which are IFNalfa-2b and pegylated IFN alfa-2a and six nucleos(t)ide analogues (NAs) which are lamivudine (3TC), tenofovir disoproxil fumarate (TDF), adefovir (ADV), telbivudine (LdT), entecavir (ETV), and vemlidy (tenofovir alafenamide (TAF)). IFN treatment is finite, but it is known to have severe side effects, and only a small percentage of patients showed a sustained virological response, measured as loss of hepatitis B surface antigen (HBsAg). NAs are inhibitors of the HBV reverse transcriptase, profoundly reduce the viral load in vast majority of treated patients, and lead to improvement of liver function and reduced incidence of liver failure and hepatocellular carcinoma. However, the treatment of NAs is infinite (Ahmed, M. et al., Drug Discov Today (2015), 20 (5), 548-561; Zoulim, F. and Locarnini, S., Gastroenterology (2009), 137 (5), 1593-1608 e1591-1592).

HBV chronic infection is caused by persistence of covalently closed circular (ccc)DNA, which exists as an episomal form in hepatocyte nuclei. cccDNA serves as the template for viral RNA transcription and subsequent viral DNA generation. Only a few copies of cccDNA per liver cell can establish or re-initiate viral replication. Therefore, a complete cure of chronic hepatitis B will require elimination of cccDNA or permanently silencing of cccDNA. However, cccDNA is intrinsically very stable and currently available therapeutics could not eliminate cccDNA or permanently silence cccDNA (Nassal, M., Gut (2015), 64 (12), 1972-1984; Gish, R. G. et al., Antiviral Res (2015), 121, 47-58; Levrero, M. et al., J Hepatol (2009), 51 (3), 581-592.). The current SoC could not eliminate the cccDNA which are already present in the infected cells. There is an urgent need to discover and develop new anti-HBV reagents to eliminate or permanently silence cccDNA, the source of chronicity (Ahmed, M. et al., Drug Discov Today (2015), 20 (5), 548-561; Nassal, M., Gut (2015), 64 (12), 1972-1984).

### SUMMARY OF THE INVENTION

Objects of the present invention are compounds of formula (I), their manufacture, medicaments based on a compound in accordance with the invention and their production as well as the use of compounds of formula (I) as cccDNA inhibitors and for the treatment or prophylaxis of HBV infection. The compounds of formula (I) show superior anti-HBV activity. In addition, the compounds of formula (I) also show good PK profiles.

The present invention relates to a compound of formula (I)

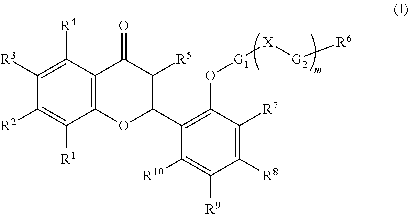

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from OH, carboxy, C₁₋₆alkoxy, C₁₋₆alkoxycarbonyl, carboxycarbonylamino and C₁₋₆alkoxycarbonylcarbonylamino;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl, C₁₋₆alkoxy and haloC₁₋₆alkoxy;
R⁹ is selected from H, OH, halogen, C₁₋₆alkyl, haloC₁₋₆alkyl and C₁₋₆alkoxy;

or R⁸ and R⁹, together with the atoms to which they are attached, form a heterocyclyl ring;
R¹⁰ is selected from H, OH, halogen, C₁₋₆alkyl, haloC₁₋₆alkyl and C₁₋₆alkoxy;
G₁ is selected from C₁₋₆alkyl and C₃₋₇cycloalkyl; wherein C₁₋₆alkyl is unsubstituted or substituted by C₁₋₆alkylsulfonylamino, C₃₋₇cycloalkylsulfonylamino, aminosulfonylamino or C₁₋₆alkylaminosulfonylamino;
X is selected from O and S;
G₂ is selected from C₁₋₆alkyl and C₃₋₇cycloalkyl;
m is selected from 0 and 1;
or a pharmaceutically acceptable salt thereof.

### DETAILED DESCRIPTION OF THE INVENTION

### Definitions

As used herein, the term “C₁₋₆alkyl” alone or in combination signifies a saturated, linear- or branched chain alkyl group containing 1 to 6, particularly 1 to 4 carbon atoms, for example methyl, ethyl, propyl, isopropyl, butyl, isobutyl, tert-butyl and the like. Particular “C₁₋₆alkyl” groups are methyl, ethyl, propyl, isopropyl, isobutyl and tert-butyl. Most particular “C₁₋₆alkyl” group is methyl.

The term “C₁₋₆alkoxy” alone or in combination signifies a group C₁₋₆alkyl-O—, wherein the “C₁₋₆alkyl” is as defined above; for example, methoxy, ethoxy, propoxy, iso-propoxy, n-butoxy, iso-butoxy, 2-butoxy, tert-butoxy, pentoxy, hexyloxy and the like. Particular “C₁₋₆alkoxy” groups are methoxy and ethoxy.

The term “C₃₋₇cycloalkyl” denotes to a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” group is cyclobutyl.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group is replaced by same or different halogen atoms, particularly fluoro atoms. Examples of haloC₁₋₆alkyl include monochloro-, difluoro- or trifluoro-methyl, -ethyl or -propyl, for example difluoromethyl and trifluoromethyl.

The term “haloC₁₋₆alkoxy” denotes a C₁₋₆alkoxy group wherein at least one of the hydrogen atoms of the C₁₋₆alkoxy group is replaced by same or different halogen atoms, particularly fluoro atoms. Examples of haloC₁₋₆alkoxy include monofluoro-, difluoro- or trifluoro-methoxy, -ethoxy or -propoxy, for example trifluoromethoxy.

“heterocyclyl” refers to any mono-, bi-, tricyclic or spiro, saturated or unsaturated, aromatic (heteroaryl) or non-aromatic (e.g., heterocycloalkyl), ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocyclyl, regardless of the point of attachment of the cyclic system to the rest of the molecule. In one example, heterocyclyl includes 3-11 ring atoms (“members”) and includes monocycles, bicycles, tricycles and spiro ring systems, wherein the ring atoms are carbon, where at least one atom in the ring or ring system is a heteroatom selected from nitrogen, sulfur or oxygen. In one example, heterocyclyl includes 3- to 7-membered monocycles having 1, 2, 3 or 4 heteroatoms selected from nitrogen, sulfur or oxygen. In another example, heterocyclyl includes 4-, 5- or 6-membered monocycles having 1, 2, 3 or 4 heteroatoms selected from nitrogen, sulfur or oxygen. In one example, heterocyclyl includes 8- to 12-membered bicycles having 1, 2, 3, 4, 5 or 6 heteroatoms selected from nitrogen, sulfur or oxygen. In another example, heterocyclyl includes 9- or 10-membered bicycles having 1, 2, 3, 4, 5 or 6 heteroatoms selected from nitrogen, sulfur or oxygen. Examplary heterocyclyls are 1,3-dioxole and 1,3-dioxolanefuryl.

The term “carbonyl” alone or in combination refers to the group —C(O)—.

The term “sulfonyl” alone or in combination refers to the group —S(O)₂—.

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of formula (I) and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435. Particular are the sodium salts of the compounds of formula (I).

Compounds of the general formula (I) which contain one or several chiral centers can either be present as racemates, diastereomeric mixtures, or optically active single isomers. The racemates can be separated according to known methods into the enantiomers. Particularly, diastereomeric salts which can be separated by crystallization are formed from the racemic mixtures by reaction with an optically active acid such as e.g. D- or L-tartaric acid, mandelic acid, malic acid, lactic acid or camphorsulfonic acid.

### HBV cccDNA Inhibitors

The present invention provides (i) a compound having the general formula (I):

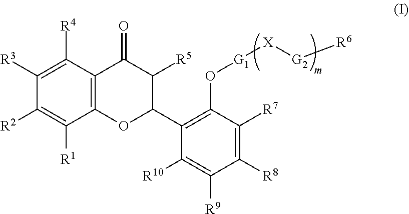

wherein
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from OH, carboxy, C₁₋₆alkoxy, C₁₋₆alkoxycarbonyl, carboxycarbonylamino and C₁₋₆alkoxycarbonylcarbonylamino;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl, C₁₋₆alkoxy and haloC₁₋₆alkoxy;
R⁹ is selected from H, OH, halogen, C₁₋₆alkyl, haloC₁₋₆alkyl and C₁₋₆alkoxy;

or R⁸ and R⁹, together with the atoms to which they are attached, form a heterocyclyl ring;
R¹⁰ is selected from H, OH, halogen, C₁₋₆alkyl, haloC₁₋₆alkyl and C₁₋₆alkoxy;
G₁ is selected from C₁₋₆alkyl and C₃₋₇cycloalkyl; wherein C₁₋₆alkyl is unsubstituted or substituted by C₁₋₆alkylsulfonylamino, C₃₋₇cycloalkylsulfonylamino, aminosulfonylamino or C₁-6alkylaminosulfonylamino;
X is selected from O and S;
G₂ is selected from C₁₋₆alkyl and C₃₋₇cycloalkyl;
m is selected from 0 and 1;
or a pharmaceutically acceptable salt thereof.

A further embodiment of the present invention is (ii) a compound of formula (I) according to (i), wherein
R¹ is halogen;
R² is selected from H, halogen and C₁₋₆alkoxy;
R³ is selected from H, halogen and C₁₋₆alkoxy;
R⁴ is selected from H and OH;
R⁵ is H;
R⁶ is selected from OH, carboxy, C₁₋₆alkoxy, C₁₋₆alkoxycarbonyl, carboxycarbonylamino and C₁₋₆alkoxycarbonylcarbonylamino;
R⁷ is selected from H;
R⁸ is selected from halogen, C₁₋₆alkyl, haloC₁₋₆alkyl, C₁₋₆alkoxy and haloC₁₋₆alkoxy;
R⁹ is selected from H, halogen, C₁₋₆alkyl and C₁₋₆alkoxy;

or R⁸ and R⁹, together with the atoms to which they are attached, form a 5-membered heterocyclyl ring;
R¹⁰ is H;
G₁ is selected from C₁₋₆alkyl and C₃₋₇cycloalkyl; wherein C₁₋₆alkyl is unsubstituted or substituted by C₁₋₆alkylsulfonylamino, aminosulfonylamino or C₁₋₆alkylaminosulfonylamino;
X is O;
G₂ is selected from C₁₋₆alkyl and C₃₋₇cycloalkyl;
m is selected from 0 and 1;
or a pharmaceutically acceptable salt thereof.

A further embodiment of the present invention is (iii) a compound of formula (I) according to (i), wherein
R¹ is Cl;
R² is selected from H, F and methoxy;
R³ is selected from H, F and methoxy;
R⁴ is selected from H and OH;
R⁵ is H;
R⁶ is selected from OH, carboxy, methoxy, methoxycarbonyl, carboxycarbonylamino and ethoxycarbonylcarbonylamino;
R⁷ is selected from H;
R⁸ is selected from Cl, Br, methyl, CF₃, methoxy and trifluoromethoxy;
R⁹ is selected from H, Br, methyl and methoxy;

or R⁸ and R⁹, together with the atoms to which they are attached, form a 5-membered heterocyclyl ring;
R¹⁰ is H;
G₁ is selected from methyl, ethyl, propyl, isobutyl and cyclobutyl; wherein ethyl is unsubstituted or substituted by ethylsulfonylamino, aminosulfonylamino or ethylaminosulfonylamino;
X is O;

G₂ is selected from methyl and cyclobutyl;
m is selected from 0 and 1;
or a pharmaceutically acceptable salt thereof.

A further embodiment of the present invention is (iv) a compound of formula (I) according to (i), or a pharmaceutically acceptable salt thereof, wherein R⁶ is carboxy.

A further embodiment of the present invention is (v) a compound of formula (I) according to (i), or a pharmaceutically acceptable salt thereof, wherein R⁸ is selected from haloC₁₋₆alkyl and haloC₁₋₆alkoxy.

A further embodiment of the present invention is (vi) a compound of formula (I) according to (i), or a pharmaceutically acceptable salt thereof, wherein R⁸ is selected from CF₃ and trifluoromethoxy.

A further embodiment of the present invention is (vii) a compound of formula (I) according to (i), or a pharmaceutically acceptable salt thereof, wherein R⁹ is selected from H and C₁₋₆alkoxy.

A further embodiment of the present invention is (viii) a compound of formula (I) according to (i), or a pharmaceutically acceptable salt thereof, wherein R⁹ is selected from H and methoxy.

A further embodiment of the present invention is (ix) a compound of formula (I) according to (i), or a pharmaceutically acceptable salt thereof, wherein G₁ is C₁₋₆alkyl.

A further embodiment of the present invention is (x) a compound of formula (I) according to (i), or a pharmaceutically acceptable salt thereof, wherein G₁ is selected from ethyl and propyl.

A further embodiment of the present invention is (xi) a compound of formula (I) according to (i), or a pharmaceutically acceptable salt thereof, wherein G₂ is C₃₋₇cycloalkyl; m is selected from 0 and 1.

A further embodiment of the present invention is (xii) a compound of formula (I) according to (i), or a pharmaceutically acceptable salt thereof, wherein G₂ is cyclobutyl; m is selected from 0 and 1.

A further embodiment of the present invention is (xiii) a compound of formula (II) according to (i), or a pharmaceutically acceptable salt thereof,

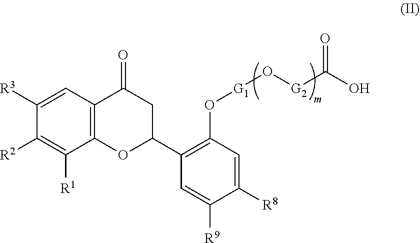

wherein
R¹ is halogen;
R² is selected from H and halogen;
R³ is selected from H and halogen;
R⁸ is selected from haloC₁₋₆alkyl and haloC₁₋₆alkoxy;
R⁹ is selected from H and C₁₋₆alkoxy;
G₁ is C₁₋₆alkyl;
G₂ is C₃₋₇cycloalkyl;
m is selected from 0 and 1.

A further embodiment of the present invention is (xiv) a compound of formula (II) according to (i), or a pharmaceutically acceptable salt thereof, wherein
R¹ is Cl;
R² is selected from H and F;
R³ is selected from H and F;
R⁸ is selected from CF₃ and trifluoromethoxy;
R⁹ is selected from H and methoxy;
G₁ is selected from ethyl and propyl;
G₂ is cyclobutyl;
m is selected from 0 and 1.

In another embodiment (xv) of the present invention, particular compounds of the present invention are selected from:

3-[2-(8-chloro-4-oxo-chroman-2-yl)-5-(trifluoromethyl)phenoxy]propanoic acid;
3-[2-(8-chloro-7-fluoro-4-oxo-chroman-2-yl)-5-(trifluoromethyl)phenoxy]propanoic acid;
3-[2-(8-chloro-6-fluoro-4-oxo-chroman-2-yl)-5-(trifluoromethyl)phenoxy]propanoic acid;
3-[2-(8-chloro-7-methoxy-4-oxo-chroman-2-yl)-5-(trifluoromethyl)phenoxy]propanoic acid;
3-[2-(8-chloro-6-methoxy-4-oxo-chroman-2-yl)-5-(trifluoromethyl)phenoxy]propanoic acid;
3-[2-(8-chloro-5-hydroxy-4-oxo-chroman-2-yl)-5-(trifluoromethyl)phenoxy]propanoic acid;
3-[5-bromo-2-(8-chloro-4-oxo-chroman-2-yl)-4-methyl-phenoxy]propanoic acid;
3-[5-bromo-2-(8-chloro-4-oxo-chroman-2-yl)-4-methoxy-phenoxy]propanoic acid;
3-[2-(8-chloro-4-oxo-chroman-2-yl)-5-methoxy-4-methyl-phenoxy]propanoic acid;
3-[5-chloro-2-(8-chloro-4-oxo-chroman-2-yl)-4-methoxy-phenoxy]propanoic acid;
3-[2-(8-chloro-4-oxo-chroman-2-yl)-4-methoxy-5-(trifluoromethyl)phenoxy]propanoic acid;
3-[[6-(8-chloro-4-oxo-chroman-2-yl)-1,3-benzodioxol-5-yl]oxy]propanoic acid;
3-[2-(8-chloro-4-oxo-chroman-2-yl)-5-(trifluoromethoxy)phenoxy]propanoic acid;
3-[5-chloro-2-(8-chloro-4-oxo-chroman-2-yl)-4-methyl-phenoxy]propanoic acid;
3-[4-bromo-2-(8-chloro-4-oxo-chroman-2-yl)-5-(trifluoromethoxy)phenoxy]propanoic acid;
3-[5-bromo-2-(8-chloro-4-oxo-chroman-2-yl)phenoxy]propanoic acid;
3-[5-bromo-2-[(2S)-8-chloro-4-oxo-chroman-2-yl]phenoxy]propanoic acid;
3-[5-bromo-2-[(2R)-8-chloro-4-oxo-chroman-2-yl]phenoxy]propanoic acid;
3-[5-chloro-2-(8-chloro-4-oxo-chroman-2-yl)-4-methyl-phenoxy]cyclobutanecarboxylic acid;
3-[2-(8-chloro-4-oxo-chroman-2-yl)-5-(trifluoromethyl)phenoxy]cyclobutanecarboxylic acid;
3-[2-(8-chloro-4-oxo-chroman-2-yl)-5-(trifluoromethyl)phenoxy]-2,2-dimethyl-propanoic acid;
3-[2-[2-(8-chloro-4-oxo-chroman-2-yl)-5-(trifluoromethyl)phenoxy]ethoxy]cyclobutanecarboxylic acid;
2-[2-[2-(8-chloro-4-oxo-chroman-2-yl)-5-(trifluoromethyl)phenoxy]ethoxy]acetic acid;
2-[3-[2-(8-chloro-4-oxo-chroman-2-yl)-5-methyl-phenoxy]propoxy]acetic acid;
2-[3-[2-(8-chloro-4-oxo-chroman-2-yl)-5-(trifluoromethyl)phenoxy]propoxy]acetic acid;
2-[3-[2-(8-chloro-7-fluoro-4-oxo-chroman-2-yl)-5-(trifluoromethyl)phenoxy]propoxy]acetic acid;
4-[2-(8-chloro-7-fluoro-4-oxo-chroman-2-yl)-5-(trifluoromethyl)phenoxy]butanoic acid;
2-[2-(8-chloro-7-fluoro-4-oxo-chroman-2-yl)-5-(trifluoromethyl)phenoxy]acetic acid;
methyl (2R)-3-[2-(8-chloro-4-oxo-chroman-2-yl)-5-(trifluoromethyl)phenoxy]-2-(ethylsulfonylamino)propanoate;
(2R)-3-[2-[(2R)-8-chloro-4-oxo-chroman-2-yl]-5-(trifluoromethyl)phenoxy]-2-(ethylsulfonylamino)propanoic acid;
(2R)-3-[2-[(2S)-8-chloro-4-oxo-chroman-2-yl]-5-(trifluoromethyl)phenoxy]-2-(ethylsulfonylamino)propanoic acid;
(2S)-3-[2-(8-chloro-4-oxo-chroman-2-yl)-5-(trifluoromethyl)phenoxy]-2-(ethylsulfonylamino)propanoic acid;
(2R)-3-[2-[(2R)-8-chloro-4-oxo-chroman-2-yl]-5-(trifluoromethyl)phenoxy]-2-(sulfamoylamino)propanoic acid;
(2R)-3-[2-[(2S)-8-chloro-4-oxo-chroman-2-yl]-5-(trifluoromethyl)phenoxy]-2-(sulfamoylamino)propanoic acid;
(2R)-3-[2-[(2S)-8-chloro-4-oxo-chroman-2-yl]-5-(trifluoromethyl)phenoxy]-2-(ethylsulfamoylamino)propanoic acid;
(2R)-3-[2-[(2R)-8-chloro-4-oxo-chroman-2-yl]-5-(trifluoromethyl)phenoxy]-2-(ethylsulfamoylamino)propanoic acid;
8-chloro-7-fluoro-2-[2-(3-methoxypropoxy)-4-(trifluoromethyl)phenyl]chroman-4-one;
8-chloro-7-fluoro-2-[2-(3-hydroxypropoxy)-4-(trifluoromethyl)phenyl]chroman-4-one;
8-chloro-7-fluoro-2-[2-(2-hydroxyethoxy)-4-(trifluoromethyl)phenyl]chroman-4-one; ethyl 2-[2-[2-(8-chloro-4-oxo-chroman-2-yl)-5-(trifluoromethyl)phenoxy]ethylamino]-2-oxo-acetate;
2-[2-[5-chloro-2-(8-chloro-4-oxo-chroman-2-yl)phenoxy]ethylamino]-2-oxo-acetic acid; and
cis-3-[5-bromo-2-(8-chloro-4-oxo-chroman-2-yl)phenoxy]cyclobutanecarboxylic acid;
or a pharmaceutically acceptable salt thereof.

In another embodiment (xvi) of the present invention, particular compounds of the present invention are selected from:

3-[2-(8-chloro-4-oxo-chroman-2-yl)-5-(trifluoromethyl)phenoxy]propanoic acid;
3-[2-(8-chloro-7-fluoro-4-oxo-chroman-2-yl)-5-(trifluoromethyl)phenoxy]propanoic acid;
3-[2-(8-chloro-6-fluoro-4-oxo-chroman-2-yl)-5-(trifluoromethyl)phenoxy]propanoic acid;
3-[2-(8-chloro-4-oxo-chroman-2-yl)-4-methoxy-5-(trifluoromethyl)phenoxy]propanoic acid;
3-[2-(8-chloro-4-oxo-chroman-2-yl)-5-(trifluoromethoxy)phenoxy]propanoic acid;
3-[2-[2-(8-chloro-4-oxo-chroman-2-yl)-5-(trifluoromethyl)phenoxy]ethoxy]cyclobutanecarboxylic acid; and
4-[2-(8-chloro-7-fluoro-4-oxo-chroman-2-yl)-5-(trifluoromethyl)phenoxy]butanoic acid;
or a pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R¹⁰, G₁, G₂, X and m are as defined above unless otherwise indicated. Furthermore, and unless explicitly otherwise stated, all reactions, reaction conditions, abbreviations and symbols have the meanings well known to a person of ordinary skill in organic chemistry.

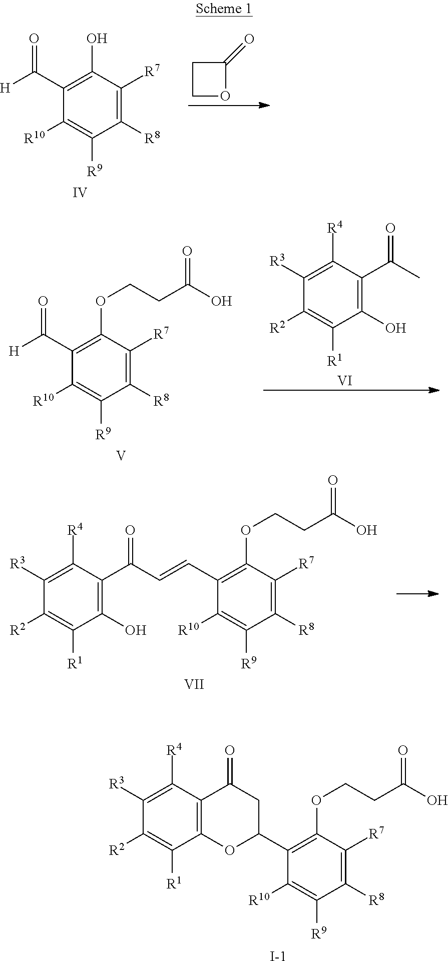

Substitution of aldehyde derivatives IV with oxetan-2-one in the presence of a suitable base, such as NaH, in a suitable solvent, such as DMF, affords compound of formula V. Condensation of compound of formula V with aryl ketone VI in the presence of a base, such as KOH, in a suitable solvent, such as ethanol, affords α,β-unsaturated carbonyl intermediates VII. Cyclization of α,β-unsaturated carbonyl intermediates VII in the presence of a suitable base, such as piperidine, in a suitable solvent, such as water, affords compound of formula I-1.

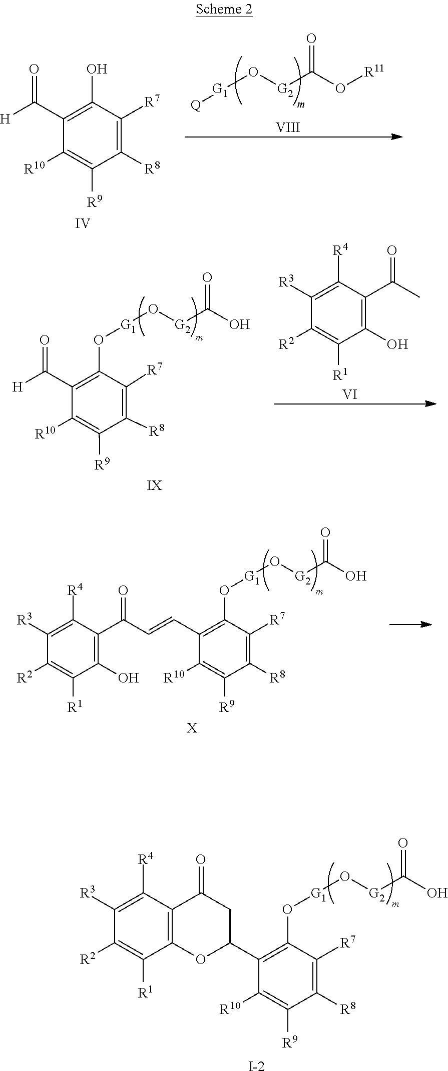

Wherein Q is halogen, OTs, OTf or OMs; R¹¹ is C₁₋₆alkyl.

Substitution of aldehyde derivatives IV with compounds of formula VIII in the presence of a suitable base, such as K₂CO₃, in a suitable solvent, such as DMF, affords compounds of formula IX. Condensation of compounds of formula IX with aryl ketone VI in the presence of a base, such as KOH, in a suitable solvent, such as ethanol, affords α, β-unsaturated carbonyl intermediate X. Cyclization of α,β-unsaturated carbonyl intermediate X in the presence of a suitable base, such as piperidine, in a suitable solvent, such as water, affords compounds of formula I-2.

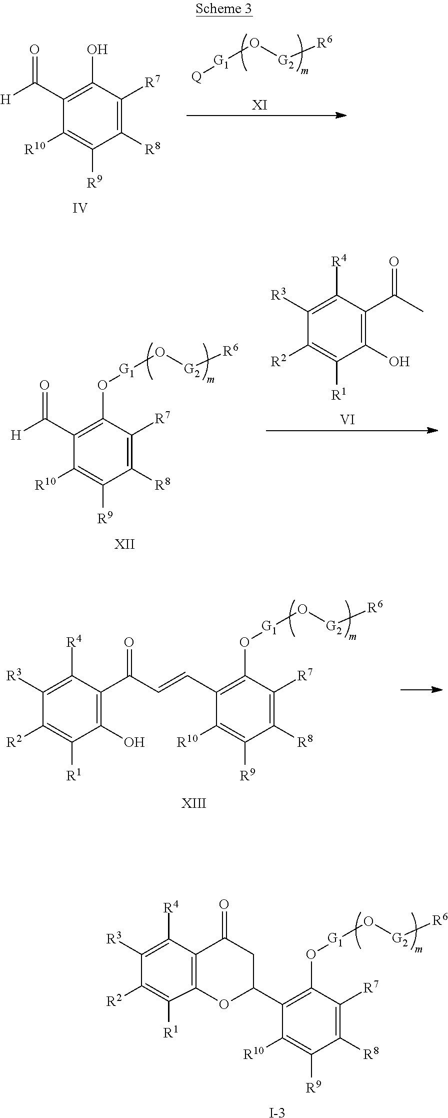

Wherein Q is halogen, OTs, OTf or Oms.

Substitution of aldehyde derivatives IV with compounds of formula XI in the presence of a suitable base, such as K₂CO₃, in a suitable solvent, such as DMF, affords compounds of formula XII. Condensation of compounds of formula XII with ketone VI in the presence of a base, such as KOH, in a suitable solvent, such as ethanol, affords α,β-unsaturated carbonyl intermediates XIII Cyclization of α,β-unsaturated carbonyl intermediates XIII in the presence of a suitable base, such as piperidine, in a suitable solvent, such as water, affords compounds of formula I-3.

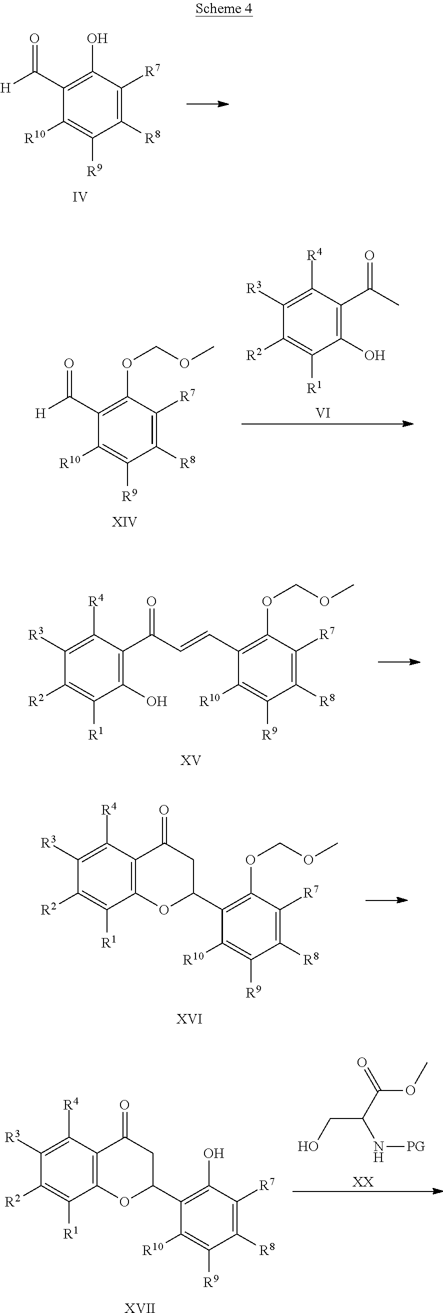

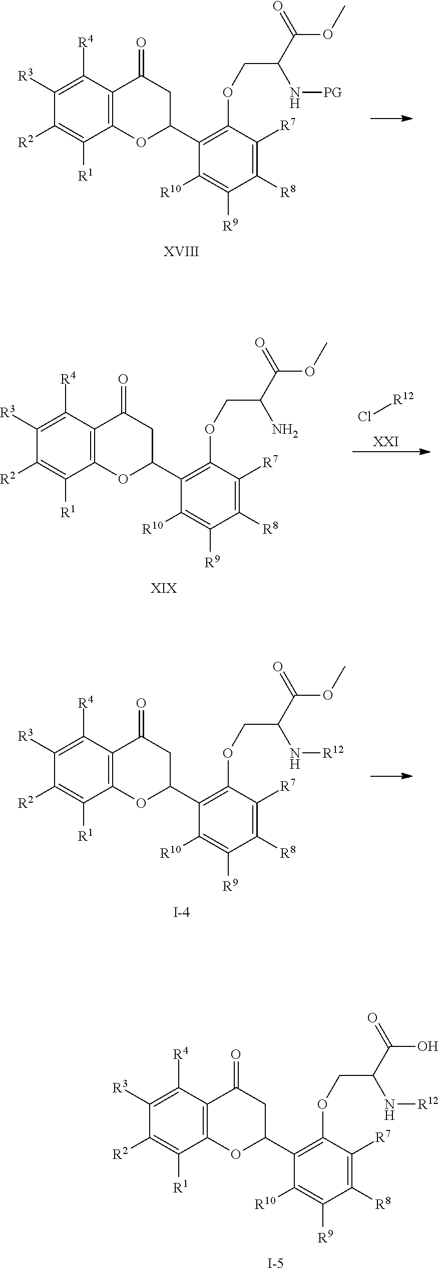

Wherein PG is triphenylmethyl; R¹² is C₁₋₆alkylsulfonyl, aminosulfonyl or C₁₋₆alkylaminosulfonyl.

Protection of hydroxy group of compounds of formula IV with bromo(methoxy)methane in the presence of a suitable base, such as NaH, in a suitable solvent, such as THF, affords compounds of formula XIV. Condensation of compounds of formula XIV with compounds of formula VI in the presence of a base, such as KOH, in a suitable solvent, such as ethanol, affords α,β-unsaturated carbonyl intermediates XV. Cyclization of α, β-unsaturated carbonyl intermediates XV in the presence of a suitable base, such as piperidine, in a suitable solvent, such as MeOH, affords compounds of formula XVI. Deprotection of compounds of formula XVI with a suitable acid, such as TFA, in a suitable solvent, such as DCM, affords compounds of formula XVII. Condensation of compounds of formula XVII with intermediates XX under Mitsunobu reaction condition affords compounds of formula XVIII. Deprotection of compounds of formula XVIII with a suitable acid, such as TFA, in a suitable solvent, such as DCM, affords compounds of formula XIX. Treatment of compounds of formula XIX with compounds of formula XXI in the presence of a suitable base, such as TEA, in a suitable solvent such as DCM, affords compounds of formula I-4. Hydrolysis of compounds of formula I-4 with a suitable base, such as trimethylstannanol, in a suitable solvent, such as DCE, affords compounds of formula I-5.

This invention also relates to a process for the preparation of a compound of formula (I) comprising at least one of the following steps:

(a) Cyclization of an α,β-unsaturated carbonyl intermediate (XIII),

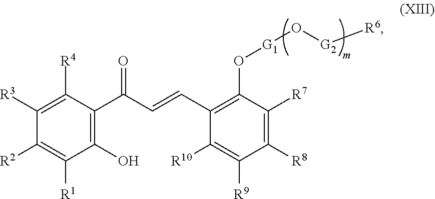

in the presence of a base;
(b) Treatment of a compound of formula (XIX),

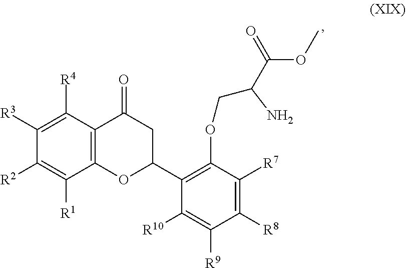

with a compound of formula (XXI),

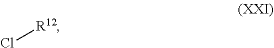

in the presence of a base;
(c) Hydrolysis of a compound of formula (I-4),

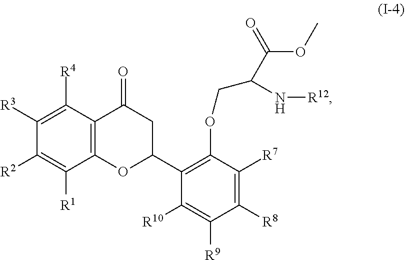

in the presence of a base;
wherein R¹ to R¹⁰, G₁, G₂ and m are defined above; R¹² is C₁₋₆alkylsulfonyl, aminosulfonyl or C₁₋₆alkylaminosulfonyl.
The base in step (a) can be for example piperidine;
The base in step (b) can be for example TEA;

The base in step (c) can be for example trimethylstannanol.

A compound of formula (I) or (II) when manufactured according to the above process is also an object of the invention.

The compound of this invention also shows good safety and PK profile.

### Pharmaceutical Compositions and Administration

The invention also relates to a compound of formula (I) or (II) for use as therapeutically active substance. Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) or (II) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) or (II) is formulated in an acetate buffer, at pH 5. In another embodiment, the compounds of formula (I) or (II) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit cccDNA in HBV patients, consequently lead to the reduction of HBsAg and HBeAg (HBV e antigen) in serum. For example, such amount may be below the amount that is toxic to normal cells, or the mammal as a whole.

In one example, the pharmaceutically effective amount of the compound of the invention administered parenterally per dose will be in the range of about 0.1 to 100 mg/kg, alternatively about 0.1 to 50 mg/kg of patient body weight per day, with the typical initial range of compound used being 0.3 to 15 mg/kg/day. In another embodiment, oral unit dosage forms, such as tablets and capsules, preferably contain from about 25 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 25 to 500 mg of the compound of the invention compounded with about 90 to 30 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of Formula (I) or (II), or pharmaceutically acceptable salt or enantiomer or diastereomer thereof.

In a further embodiment includes a pharmaceutical composition comprising a compound of formula (I) or (II), or pharmaceutically acceptable salt or enantiomer or diastereomer thereof, together with a pharmaceutically acceptable carrier or excipient.

Another embodiment includes a pharmaceutical composition comprising a compound of formula (I) or (II), or pharmaceutically acceptable salt or enantiomer or diastereomer thereof for use in the treatment of HBV infection.

### Indications and Methods of Treatment

The compounds of the invention can inhibit cccDNA and have anti-HBV activity. Accordingly, the compounds of the invention are useful for the treatment or prophylaxis of HBV infection.

The invention relates to the use of a compound of formula (I) or (II) for the inhibition of cccDNA.

The invention also relates to the use of a compound of formula (I) or (II) for the inhibition of HBeAg.

The invention further relates to the use of a compound of formula (I) or (II) for the inhibition of HBsAg.

The invention relates to the use of a compound of formula (I) or (II) for the inhibition of HBV DNA.

The invention relates to the use of a compound of formula (I) or (II) for use in the treatment or prophylaxis of HBV infection.

The use of a compound of formula (I) or (II) for the preparation of medicaments useful in the treatment or prophylaxis diseases that are related to HBV infection is an object of the invention.

The invention relates in particular to the use of a compound of formula (I) or (II) for the preparation of a medicament for the treatment or prophylaxis of HBV infection.

Another embodiment includes a method for the treatment or prophylaxis of HBV infection, which method comprises administering an effective amount of a compound of Formula (I) or (II), or enantiomers, diastereomers, prodrugs or pharmaceutically acceptable salts thereof.

### EXAMPLES

The invention will be more fully understood by reference to the following examples. They should not, however, be construed as limiting the scope of the invention.

Abbreviations used herein are as follows:
ACN: acetonitrile
BBr3: boron tribromide
DMAP: 4-dimethylaminopyridine

### DMF: N,N-dimethylformamide

IC50: the molar concentration of an inhibitor, which produces 50% of the maximum possible response for that inhibitor.
FBS: fetal bovine serum
HPLC: high performance liquid chromatography
MS (ESI): mass spectroscopy (electron spray ionization)
Ms: methylsulfonyl
obsd.: observed
PE: petroleum ether
EtOAc: ethyl acetate
AcOH: acetic acid
THF: tetrahydrofuran
TFA: trifluoroacetic acid

### DIPEA: N,N-Diisopropylethylamine

DIAD: Diisopropyl azodicarboxylate
Ts: p-tolylsulfonyl
δ: chemical shift

### General Experimental Conditions

Intermediates and final compounds were purified by flash chromatography using one of the following instruments: i) Biotage SP1 system and the Quad 12/25 Cartridge module. ii) column chromatography on silica gel combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C18 (5 μm, OBD™ 30×100 mm) column or SunFire™ Perp C₁₈ (5 μm, OBD™ 30×100 mm) column.

LC/MS spectra were obtained using a Waters UPLC-SQD Mass. Standard LC/MS conditions were as follows (running time 3 minutes):

Acidic condition: A: 0.1% formic acid and 1% acetonitrile in H₂O; B: 0.1% formic acid in acetonitrile;

Basic condition: A: 0.05% NH₃.H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere. Reagents were used as received from commercial suppliers without further purification unless otherwise noted.

### PREPARATIVE EXAMPLES

### Example 1

### 3-[2-(8-chloro-4-oxo-chroman-2-yl)-5-(trifluoromethyl)phenoxy]propanoic Acid

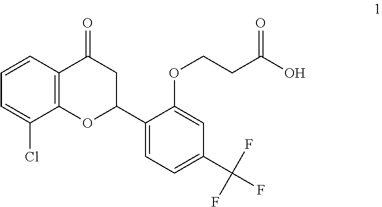 [SMILES: O=C(O)CCOc1cc(C(F)(F)F)ccc1C1CC(=O)c2cccc(Cl)c2O1]

### Step 1: Preparation of 3-[2-formyl-5-(trifluoromethyl)phenoxy]propanoic Acid

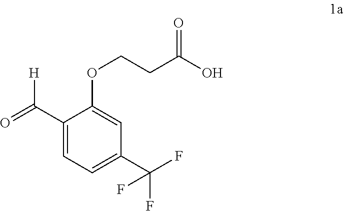 [SMILES: O=Cc1ccc(C(F)(F)F)cc1OCCC(=O)O]

To a solution of NaOH (315 mg, 7.9 mmol) in water (10 mL) was added 3-bromopropionic acid (1.27 g, 7.9 mmol) and the mixture was stirred at r.t. for 30 minutes. The resulting mixture was added into a mixture of 2-hydroxy-4-(trifluoromethyl)benzaldehyde (1500 mg, 7.9 mmol) and NaOH (315 mg, 7.9 mmol) in water (10 mL) at 100° C. dropwise and the mixture was stirred at 100° C. for additional 30 minutes. After the reaction was completed, the mixture was diluted with water (30 mL) and adjusted to pH˜2 by addition of 1N hydrochloric acid. The resulting solution was extracted with EtOAc (30 mL) three times. The combined organic layer was washed with brine (30 mL) twice, dried over Na₂SO₄ and concentrated in vacuo. The residue was purified by column chromatography on silica gel (eluting with PE:EtOAc=3:1) to give 3-[2-formyl-5-(trifluoromethyl)phenoxy]propanoic acid (600 mg, yield 29%) as a white solid. MS obsd. (ESI⁺) [(M+H)⁺]:263.1.

### Step 2: Preparation of 3-[2-[(E)-3-(3-chloro-2-hydroxy-phenyl)-3-oxo-prop-1-enyl]-5-trifluoromethyl)phenoxy]propanoic Acid

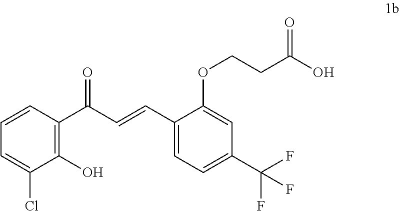 [SMILES: O=C(O)CCOc1cc(C(F)(F)F)ccc1/C=C/C(=O)c1cccc(Cl)c1O]

To a solution of 3-[2-formyl-5-(trifluoromethyl)phenoxy]propanoic acid (200.0 mg, 0.76 mmol) and 1-(3-chloro-2-hydroxy-phenyl)ethanone (130.0 mg, 0.76 mmol) in ethanol (10 mL) was added KOH (428.0 mg, 7.6 mmol). The mixture was stirred at 35° C. for 16 hours. After the reaction was completed, the reaction mixture was diluted with water (50 mL) and acidified to pH˜1.0 by addition of 1N HCl. The resulting suspension was filtered. The filter cake was collected and dried in vacuo to give the crude of 3-[2-[(E)-3-(3-chloro-2-hydroxy-phenyl)-3-oxo-prop-1-enyl]-5-(trifluoromethyl)phenoxy]propanoic acid (210.0 mg, 63.7% yield) as a yellow solid, which was used in the next step directly. MS obsd. (ESI⁺) [(M+H)⁺]:415.0.

### Step 3: Preparation of 3-[2-(8-chloro-4-oxo-chroman-2-yl)-5-(trifluoromethyl)phenoxy]propanoic Acid

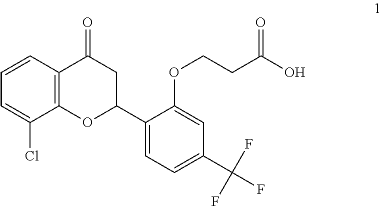 [SMILES: O=C(O)CCOc1cc(C(F)(F)F)ccc1C1CC(=O)c2cccc(Cl)c2O1]

To a solution of 3-[2-[(E)-3-(3-chloro-2-hydroxy-phenyl)-3-oxo-prop-1-enyl]-5-(trifluoromethyl)phenoxy]propanoic acid (120.0 mg, 0.290 mmol) in water (3 mL) were added potassium hydroxide (32.47 mg, 0.580 mmol) and piperidine (0.74 mg, 0.010 mmol). The mixture was then stirred at 25° C. for 2 hours. After the reaction was completed, the mixture was diluted with water (30 mL) and adjusted to pH˜2 by addition of 1N HCl. The resulting mixture was then extracted with EtOAc (30 mL) three times. The combined organic layer was washed with brine (30 mL), dried over Na₂SO₄ and concentrated in vacuo. The residue was purified by preparative HPLC to give 3-[2-(8-chloro-4-oxo-chroman-2-yl)-5-(trifluoromethyl)phenoxy]propanoic acid (33.8 mg 28.11% yield) as a yellow solid. ¹H NMR (DMSO-d₆, 400 MHz): δ ppm 12.39 (s, 1H), 7.75-7.85 (m, 3H), 7.48 (d, J=7.9 Hz, 1H), 7.44 (s, 1H), 7.14 (t, J=7.8 Hz, 1H), 5.93 (d, J=10.8 Hz, 1H), 4.34 (dtd, J=15.3, 9.9, 5.8 Hz, 2H), 3.19 (dd, J=16.9, 13.3 Hz, 1H), 2.89 (dd, J=16.9, 2.7 Hz, 1H), 2.73 (t, J=5.7 Hz, 2H). MS obsd. (ESI⁺) [(M+H)⁺]:415.1.

### Example 2

### 3-[2-(8-chloro-7-fluoro-4-oxo-chroman-2-yl)-5-(trifluoromethyl)phenoxy]propanoic Acid

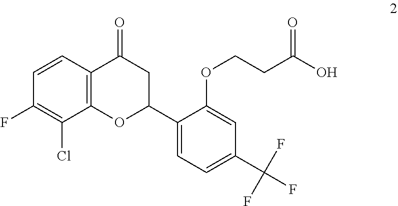 [SMILES: O=C(O)CCOc1cc(C(F)(F)F)ccc1C1CC(=O)c2ccc(F)c(Cl)c2O1]

### Step 1: Preparation of (2-chloro-3-fluoro-phenyl) acetate

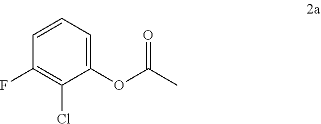

To a mixture of 2-chloro-3-fluoro-phenol (10.0 g, 68.9 mmol) and TEA (7.6 g, 75.06 mmol) in dichloromethane (150 mL) was added acetyl chloride (5.36 g, 68.24 mmol) at 0° C. and the mixture was then stirred at room temperature for 16 hours. After the reaction was completed, the mixture was poured into water (30 mL) and extracted with dichloromethane (50 mL) three times. The combined organic layer was washed with brine, dried over anhydrous Na₂SO₄ and concentrated in vacuo. The residue was then purified by column chromatography on silica gel (eluting with PE:EtOAc=50:1˜20:1) to give (2-chloro-3-fluoro-phenyl) acetate (10.0 g, 77.2% yield) as colorless oil. MS obsd. (ESI⁺) [(M+H)⁺]: 188.1.

### Step 2: Preparation of 1-(3-chloro-4-fluoro-2-hydroxy-phenyl)ethanone

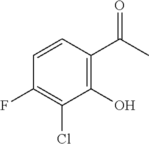

A mixture of (2-chloro-3-fluoro-phenyl) acetate (10.0 g, 53.1 mmol) and AlCl₃ (7.07 g, 53.03 mmol) was stirred at 150° C. for 5 hours. After the reaction was completed, the mixture was poured into water (100 mL) and extracted with EtOAc (250 mL) twice. The combined organic layer was washed with brine, dried over anhydrous Na₂SO₄ and concentrated in vacuo. The residue was then purified by column chromatography on silica gel (eluting with PE:EtOAc=50:1˜20:1) to give 1-(3-chloro-4-fluoro-2-hydroxy-phenyl)ethanone (3.0 g, 30.0% yield) as a white solid. MS obsd. (ESI⁺) [(M+H)⁺]: 188.2.

### Step 3: Preparation of 3-[2-(8-chloro-7-fluoro-4-oxo-chroman-2-yl)-5-(trifluoromethyl)phenoxy]propanoic Acid

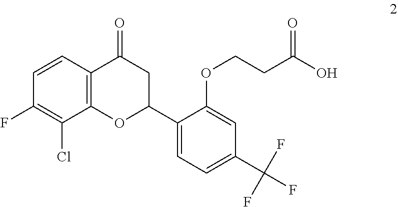 [SMILES: O=C(O)CCOc1cc(C(F)(F)F)ccc1C1CC(=O)c2ccc(F)c(Cl)c2O1]

Example 2 was prepared in analogy to the procedure described for the preparation of example 1 by using 1-(3-chloro-4-fluoro-2-hydroxy-phenyl)ethanone as the starting material instead of 1-(3-chloro-2-hydroxy-phenyl)ethanone in Step 2. ¹H NMR (DMSO-d₆, 400 MHz): δ ppm 12.43 (s, 1H), 7.76-7.90 (m, 2H), 7.48 (d, J=8.1 Hz, 1H), 7.45 (s, 1H), 7.22 (t, J=8.8 Hz, 1H), 6.00 (dd, J=13.2, 2.7 Hz, 1H), 4.19-4.46 (m, 2H), 3.20 (dd, J=16.9, 13.2 Hz, 1H), 2.89 (dd, J=16.9, 2.9 Hz, 1H), 2.72 (t, J=5.8 Hz, 2H). MS obsd. (ESI⁺) [(M+H)⁺]:433.0.

### Example 3

### 3-[2-(8-chloro-6-fluoro-4-oxo-chroman-2-yl)-5-(trifluoromethyl)phenoxy]propanoic Acid

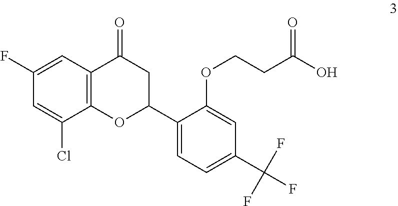 [SMILES: O=C(O)CCOc1cc(C(F)(F)F)ccc1C1CC(=O)c2cc(F)cc(Cl)c2O1]

### Step 1: Preparation of 1-(3-chloro-5-fluoro-2-hydroxy-phenyl)ethanone

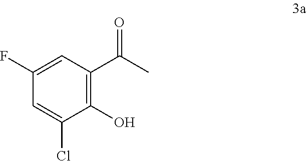

Compound 3a was prepared in analogy to the procedure described for the preparation of compound 2b by using 2-chloro-4-fluoro-phenol as the starting materials instead of 2-chloro-3-fluoro-phenol in Step 1. MS obsd. (ESI⁺) [(M+H)⁺]: 188.1.

### Step 2: Preparation of 3-[2-(8-chloro-6-fluoro-4-oxo-chroman-2-yl)-5-(trifluoromethyl)phenoxy]propanoic Acid

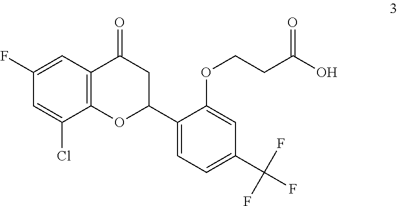 [SMILES: O=C(O)CCOc1cc(C(F)(F)F)ccc1C1CC(=O)c2cc(F)cc(Cl)c2O1]

Example 3 was prepared in analogy to the procedure described for the preparation of example 1 by using 1-(3-chloro-5-fluoro-2-hydroxy-phenyl)ethanone as the starting material instead of 1-(3-chloro-2-hydroxy-phenyl)ethanone in Step 2. ¹H NMR (DMSO-d₆, 400 MHz): δ ppm 12.47 (s, 1H), 7.72-7.98 (m, 2H), 7.34-7.58 (m, 3H), 5.94 (dd, J=13.3, 2.6 Hz, 1H), 4.26-4.41 (m, 2H), 3.20 (dd, J=17.0, 13.4 Hz, 1H), 2.92 (dd, J=17.0, 2.9 Hz, 1H), 2.72 (t, J=5.8 Hz, 2H). MS obsd. (ESI⁺) [(M+H)⁺]:433.0.

### Example 4

### 3-[2-(8-chloro-7-methoxy-4-oxo-chroman-2-yl)-5-(trifluoromethyl)phenoxy]propanoic Acid

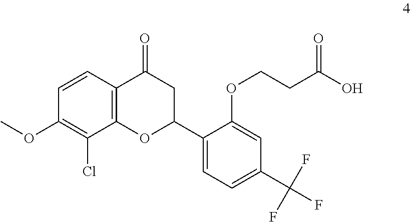 [SMILES: COc1ccc2c(c1Cl)OC(c1ccc(C(F)(F)F)cc1OCCC(=O)O)CC2=O]

### Step 1: Preparation of 1-(3-chloro-2-hydroxy-4-methoxy-phenyl)ethanone

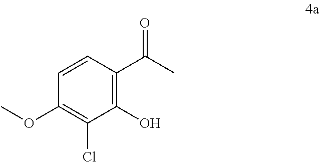

Compound 4a was prepared in analogy to the procedure described for the preparation of compound 2b by using 2-chloro-3-methoxy-phenol as the starting materials instead of 2-chloro-3-fluoro-phenol in Step 1. MS obsd. (ESI⁺) [(M+H)⁺]: 200.2.

### Step 2: Preparation of 3-[2-(8-chloro-7-methoxy-4-oxo-chroman-2-yl)-5-(trifluoromethyl)phenoxy]propanoic Acid

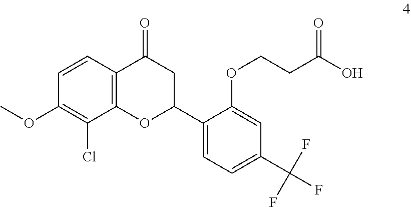 [SMILES: COc1ccc2c(c1Cl)OC(c1ccc(C(F)(F)F)cc1OCCC(=O)O)CC2=O]

Example 4 was prepared in analogy to the procedure described for the preparation of example 1 by using 1-(3-chloro-2-hydroxy-4-methoxy-phenyl)ethanone as the starting material instead of 1-(3-chloro-2-hydroxy-phenyl)ethanone in Step 2. ¹H NMR (DMSO-d₆, 400 MHz): δ ppm 12.44 (s, 1H), 7.80 (d, J=8.9 Hz, 1H), 7.47 (d, J=8.2 Hz, 2H), 7.43 (s, 1H), 7.00 (d, J=9.0 Hz, 1H), 5.88 (dd, J=12.8, 2.8 Hz, 1H), 4.17— 4.49 (m, 2H), 3.95 (s, 3H), 3.08 (dd, J=16.9, 12.9 Hz, 1H), 2.84 (dd, J=16.9, 3.0 Hz, 1H), 2.72 (t, J=5.8 Hz, 2H). MS obsd. (ESI⁺) [(M+H)⁺]:445.0.

### Example 5

3-[2-(8-chloro-6-methoxy-4-oxo-chroman-2-yl)-5-(trifluoromethyl)phenoxy]propanoic Acid

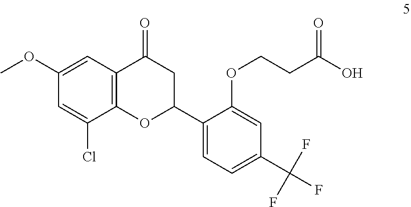 [SMILES: COc1cc(Cl)c2c(c1)C(=O)CC(c1ccc(C(F)(F)F)cc1OCCC(=O)O)O2]

### Step 1: Preparation of 1-(3-chloro-2-hydroxy-5-methoxy-phenyl)ethanone

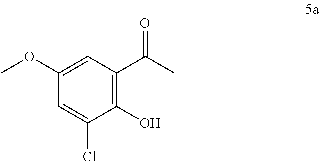

Compound 5a was prepared in analogy to the procedure described for the preparation of compound 2b by using 2-chloro-4-methoxy-phenol as the starting materials instead of 2-chloro-3-fluoro-phenol in Step 1. MS obsd. (ESI⁺) [(M+H)⁺]: 200.2.

### Step 2: Preparation of 3-[2-(8-chloro-6-methoxy-4-oxo-chroman-2-yl)-5-(trifluoromethyl)phenoxy]propanoic Acid

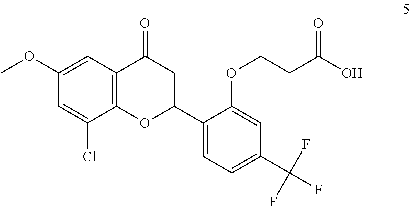 [SMILES: COc1cc(Cl)c2c(c1)C(=O)CC(c1ccc(C(F)(F)F)cc1OCCC(=O)O)O2]

Example 5 was prepared in analogy to the procedure described for the preparation of example 1 by using 1-(3-chloro-2-hydroxy-5-methoxy-phenyl)ethanone as the starting material instead of 1-(3-chloro-2-hydroxy-phenyl)ethanone in Step 2. ¹H NMR (DMSO-d₆, 400 MHz): δ ppm 12.43 (s, 1H), 7.80 (d, J=8.0 Hz, 1H), 7.45 — 7.49 (m, 2H), 7.43 (s, 1H), 7.23 (d, J=3.1 Hz, 1H), 5.84 (dd, J=13.2, 2.6 Hz, 1H), 4.26 — 4.41 (m, 2H), 3.80 (s, 3H), 3.14 (dd, J=17.0, 13.2 Hz, 1H), 2.89 (dd, J=17.0, 2.9 Hz, 1H), 2.72 (t, J=5.8 Hz, 2H). MS obsd. (ESI⁺) [(M+H)⁺]:445.1.

### Example 6

### 3-[2-(8-chloro-5-hydroxy-4-oxo-chroman-2-yl)-5-(trifluoromethyl)phenoxy]propanoic Acid

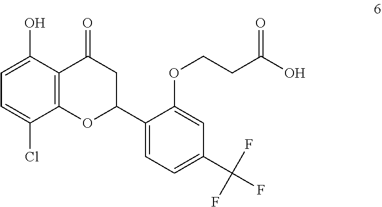 [SMILES: O=C(O)CCOc1cc(C(F)(F)F)ccc1C1CC(=O)c2c(O)ccc(Cl)c2O1]

### Step 1: Preparation of 1-(3-chloro-2,6-dihydroxy-phenyl)ethanone

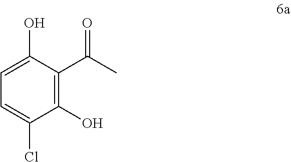

A mixture of 1-(2,6-dihydroxyphenyl)ethan-1-one (5.0 g, 32.9 mmol), 1-chloropyrrolidine-2,5-dione (5.27 g, 39.4 mmol) in acetic acid (25 mL) was stirred at 50° C. for 2 hours. After the reaction was completed, the mixture was concentrated in vacuo, the residue was triturated with EtOAc (15 mL) and the suspension was then filtered. The filtrate was concentrated in vacuo, the residue was purified by column chromatography on silica gel (eluting with PE:EtOAc=100:1 to 3:1) to give 1-(3-chloro-2,6-dihydroxy-phenyl)ethanone (5.0 g, 81.5% yield) as a yellow solid.

### Step 2: Preparation of 1-[3-chloro-6-hydroxy-2-[(4-methoxyphenyl)methoxy]phenyl]ethenone

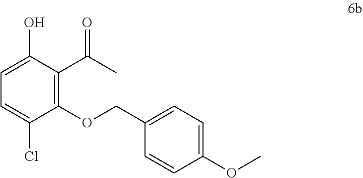 [SMILES: COc1ccc(COc2c(Cl)ccc(O)c2C(C)=O)cc1]

To a solution of 1-(3-chloro-2,6-dihydroxy-phenyl)ethanone (920.0 mg, 4.93 mmol) in DMF (15 mL) were added 4-methoxybenzylchloride (0.67 mL, 4.93 mmol) and K₂CO₃ (681.46 mg, 4.93 mmol). The reaction mixture was stirred at 20° C. for 16 hours. After the reaction was completed, the mixture was concentrated in vacuo and the residue was purified by column chromatography on silica gel (eluting with PE:EtOAc=10:1) to give 1-[3-chloro-6-hydroxy-2-[(4-methoxyphenyl)methoxy]phenyl]ethanone (720 mg, 47.61% yield) as light yellow solid. MS obsd. (ESI⁺)[(M+Na)⁺]: 329.0.

### Step 3: Preparation of 1-[3-chloro-6-(methoxymethoxy)-2-[(4-methoxyphenyl)methoxy]phenyl]ethenone

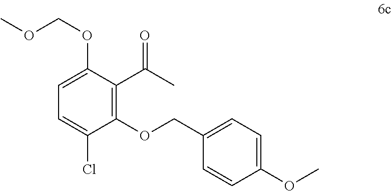

To a solution of 1-[3-chloro-6-hydroxy-2-[(4-methoxyphenyl)methoxy]phenyl]ethanone (700.0 mg, 2.28 mmol) in THF (10 mL) were added NaH (82.15 mg, 3.42 mmol) and bromomethyl methyl ether (427.7 mg, 3.42 mmol). The reaction mixture was stirred at 0° C. for 0.5 hour. The mixture was poured into water (10 mL) and the resulting mixture was extracted with EtOAc (20 mL) three times. The combined organic layer was washed with brine, dried over Na₂SO_{4,} concentrated in vacuo. The residue was purified by column chromatography on silica gel (eluting with PE:EtOAc=10:1) to give the 1-[3-chloro-6-(methoxymethoxy)-2-[(4-methoxyphenyl)methoxy]phenyl]ethanone (700 mg, 87.44% yield) as colorless oil. MS obsd. (ESI⁺)[(M+Na)⁺]: 373.1.

### Step 4: Preparation of 1-[3-chloro-2-hydroxy-6-(methoxymethoxy)phenyl]ethenone

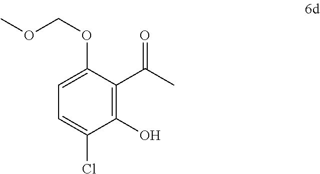

To a solution of 1-[3-chloro-6-(methoxymethoxy)-2-[(4-methoxyphenyl)methoxy]phenyl]ethanone (500.0 mg, 1.43 mmol) in DCM (15 mL) and water (1.5 mL) was added 4,5-dichloro-3,6-dihydroxy-phthalonitrile (685.51 mg, 2.99 mmol). The reaction was stirred at 25° C. for 16 hours. After the reaction was completed, the mixture was concentrated in vacuo and the residue was purified by preparative HPLC to give 1-[3-chloro hydroxy-6-(methoxymethoxy)phenyl]ethanone (322 mg, 97.95% yield) as a yellow solid.

### Step 5: Preparation of 3-[2-[(E)-3-[3-chloro-2-hydroxy-6-(methoxymethoxy)phenyl]-3-oxo-prop-1-enyl]-5-(trifluoromethyl)phenoxy]propanoic Acid

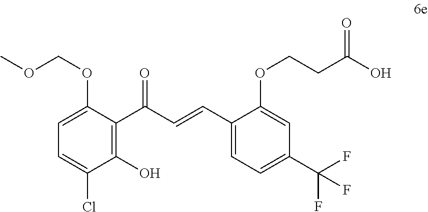 [SMILES: COCOc1ccc(Cl)c(O)c1C(=O)/C=C/c1ccc(C(F)(F)F)cc1OCCC(=O)O]

To a solution of 3[2-formyl-5-(trifluoromethyl)phenoxy]propanoic acid (242.85 mg, 0.930 mmol) and 1-[3-chloro-2-hydroxy-6-(methoxymethoxy)phenyl]ethanone (235.0 mg, 1.02 mmol) in ethanol (10 mL) was added NaOH (370.51 mg, 9.26 mmol). The mixture was stirred at r.t. for 16 hours. After the reaction was completed, the reaction mixture was adjusted to pH˜6 by addition of 1N HCl. The resulting suspension was filtered, the solid was collected and dried in vacuo to give 3-[2-[(E)-3-[3-chloro-2-hydroxy-6-(methoxymethoxy)phenyl]-3-oxo-prop-1-enyl]-5-(trifluoromethyl)phenoxy]propanoic acid (160 mg, 0.340 mmol) as a yellow solid. MS obsd. (ESI⁺)[(M+H)⁺]: 497.1.

### Step 6: Preparation of 3-[2-[8-chloro-5-(methoxymethoxy)-4-oxo-chroman-2-yl]-5-(trifluoromethyl)phenoxy]propanoic Acid

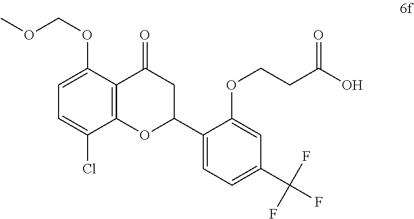 [SMILES: COCOc1ccc(Cl)c2c1C(=O)CC(c1ccc(C(F)(F)F)cc1OCCC(=O)O)O2]

A solution of 3-[2-[(E)-3-[3-chloro-2-hydroxy-6-(methoxymethoxy)phenyl]-3-oxo-prop-1-enyl]-5-(trifluoromethyl)phenoxy]propanoic acid (100.0 mg, 0.210 mmol) and pyridine (10.0 mL, 123.64 mmol) in the mixed solvent of methanol (10 mL) and water (10 mL) was stirred at 90° C. for 16 hours. After the reaction was completed, the reaction mixture was concentrated in vacuo to give the crude of 3-[2-[8-chloro-5-(methoxymethoxy)-4-oxo-chroman-2-yl]-5-(trifluoromethyl)phenoxy]propanoic acid (80 mg, 80% yield) as a yellow solid, which was used in next step directly without further purification. MS obsd. (ESI⁺)[(M+Na)⁺]: 497.0.

### Step 7: Preparation of 3-[2-(8-chloro-5-hydroxy-4-oxo-chroman-2-yl)-5-(trifluoromethyl)phenoxy]propanoic Acid

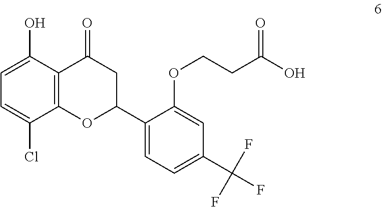 [SMILES: O=C(O)CCOc1cc(C(F)(F)F)ccc1C1CC(=O)c2c(O)ccc(Cl)c2O1]

To a solution of 3-[2-[8-chloro-5-(methoxymethoxy)-4-oxo-chroman-2-yl]-5-(trifluoromethyl)phenoxy]propanoic acid (50.0 mg, 0.110 mmol) in DCM (2.00 mL) was added TFA (0.5 mL, 25.96 mmol) and the mixture was stirred at r.t. for 3 hours. After the reaction was completed, the mixture was concentrated in vacuo. The residue was purified by preparative HPLC to give the 3-[2-(8-chloro-5-hydroxy-4-oxo-chroman-2-yl)-5-(trifluoromethyl)phenoxy]propanoic acid (12 mg, 25.93% yield) as a white solid. ¹H NMR (400 MHz, DMSO-d₆): δ ppm 12.48 (s, 1H), 11.68 (s, 1H), 7.79 (d, J=8.1 Hz, 1H), 7.62-7.69 (m, 1H), 7.42 —7.51 (m, 2H), 6.61 (d, J=8.9 Hz, 1H), 5.96 (dd, J=13.2, 2.7 Hz, 1H), 4.22— 4.47 (m, 2H), 3.28 (s, 1H), 2.94 (dd, J=17.2, 3.0 Hz, 1H), 2.74 (t, J=5.8 Hz, 2H). MS obsd. (ESI⁺)[(M+H)⁺]: 431.0.

### Example 7

### 3-[5-bromo-2-(8-chloro-4-oxo-chroman-2-yl)-4-methyl-phenoxy]propanoic Acid

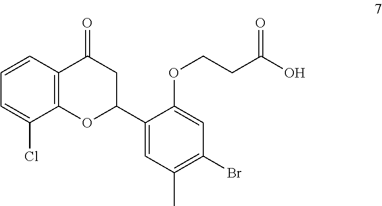 [SMILES: Cc1cc(C2CC(=O)c3cccc(Cl)c3O2)c(OCCC(=O)O)cc1Br]

### Step 1: Preparation of 4-bromo-2-hydroxy-5-methyl-benzaldehyde

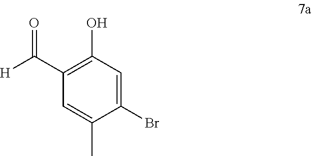

To a solution of 3-bromo-4-methyl-phenol (10.0 g, 53.4 mmol) in ACN (200 mL) were added formaldehyde (8.42 g, 280.54 mmol), TEA (39.1 mL, 280.5 mmol) and magnesium chloride (27.0 mL, 210.4 mmol) and the mixture was stirred at 80° C. for 16 hours. After the reaction was completed, the mixture was quenched with 1M HCl (500 mL) and extracted with EtOAc (150 mL) three times. The combined organic layer was washed with brine, dried over Na₂SO₄ and concentrated in vacuo to give the crude of 4-bromo-2-hydroxy-5-methyl-benzaldehyde (10.3 g, 87.9% yield) as brown oil, which was used in the next step directly without further purification.

### Step 2: Preparation of 3-[5-bromo-2-(8-chloro-4-oxo-chroman-2-yl)-4-methyl-phenoxy]propanoic Acid

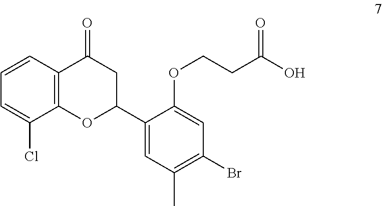 [SMILES: Cc1cc(C2CC(=O)c3cccc(Cl)c3O2)c(OCCC(=O)O)cc1Br]

Example 7 was prepared in analogy to the procedure described for the preparation of example 1 by using 4-bromo-2-hydroxy-5-methyl-benzaldehyde as the starting material instead of 2-hydroxy-4-(trifluoromethyl)benzaldehyde in Step 1. ¹H NMR (DMSO-d₆, 400 MHz): δ ppm 12.38 (s, 1H), 7.77 (ddd, J=7.7, 5.7, 1.6 Hz, 2H), 7.56 (s, 1H), 7.36 (s, 1H), 7.12 (t, J=7.8 Hz, 1H), 5.80 (dd, J=13.6, 2.6 Hz, 1H), 4.16-4.29 (m, 2H), 3.23 (dd, J=16.9, 13.5 Hz, 1H), 2.78 (dd, J=16.9, 2.8 Hz, 1H), 2.66 (t, J=6.0 Hz, 2H), 2.31 (s, 3H). MS obsd. (ESI⁺) [(M+H)⁺]:439.0.

### Example 8

### 3-[5-bromo-2-(8-chloro-4-oxo-chroman-2-yl)-4-methoxy-phenoxy]propanoic Acid

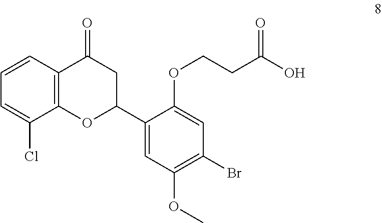 [SMILES: COc1cc(C2CC(=O)c3cccc(Cl)c3O2)c(OCCC(=O)O)cc1Br]

### Step 1: Preparation of 4-bromo-2-hydroxy-5-methoxy-benzaldehyde

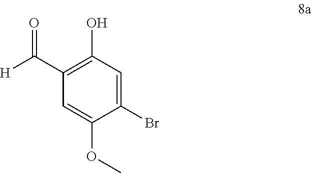

Compound 8a was prepared in analogy to the procedure described for the preparation of compound 7a by using 3-bromo-4-methoxy-phenol as the starting material instead of 3-bromo-4-methyl-phenol.

### Step 2: Preparation of 3-[5-bromo-2-(8-chloro-4-oxo-chroman-2-yl)-4-methoxy-phenoxy]propanoic Acid

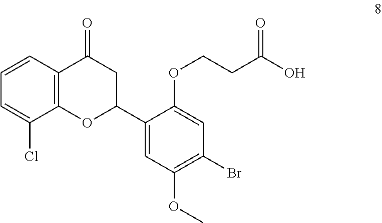 [SMILES: COc1cc(C2CC(=O)c3cccc(Cl)c3O2)c(OCCC(=O)O)cc1Br]

Example 8 was prepared in analogy to the procedure described for the preparation of example 1 by using 4-bromo-2-hydroxy-5-methoxy-benzaldehyde as the starting material instead of 2-hydroxy-4-(trifluoromethyl)benzaldehyde in Step 1. ¹H NMR (DMSO-d₆, 400 MHz): δ ppm 12.36 (s, 1H), 7.78 (ddd, J=7.8, 5.9, 1.6 Hz, 2H), 7.41 (s, 1H), 7.36 (s, 1H), 7.13 (t, J=7.8 Hz, 1H), 5.86 (dd, J=13.6, 2.6 Hz, 1H), 4.13-4.25 (m, 2H), 3.83 (s, 3H), 3.36 (d, J=13.6 Hz, 1H), 2.79 (dd, J=16.9, 2.8 Hz, 1H), 2.65 (t, J=5.9 Hz, 2H). MS obsd. (ESI⁺) [(M+H)⁺]:455.0.

### Example 9

3-[2-(8-chloro-4-oxo-chroman-2-yl)-5-methoxy-4-methyl-phenoxy]propanoic Acid

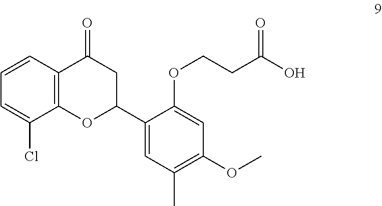 [SMILES: COc1cc(OCCC(=O)O)c(C2CC(=O)c3cccc(Cl)c3O2)cc1C]

### Step 1: Preparation of 2-hydroxy-4-methoxy-5-methyl-benzaldehyde

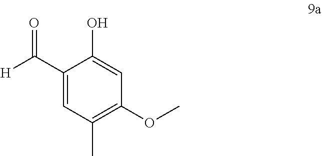

Compound 9a was prepared in analogy to the procedure described for the preparation of compound 7a by using 3-methoxy-4-methyl-phenol as the starting material instead of 3-bromo-4-methyl-phenol.

### Step 2: Preparation of 3-[2-(8-chloro-4-oxo-chroman-2-yl)-5-methoxy-4-methyl-phenoxy]propanoic Acid

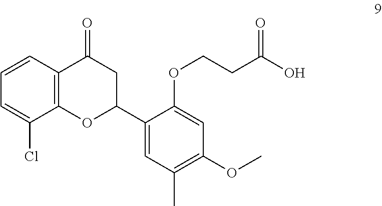 [SMILES: COc1cc(OCCC(=O)O)c(C2CC(=O)c3cccc(Cl)c3O2)cc1C]

Example 9 was prepared in analogy to the procedure described for the preparation of example 1 by using 2-hydroxy-4-methoxy-5-methyl-benzaldehyde as the starting material instead of 2-hydroxy-4-(trifluoromethyl)benzaldehyde in Step 1. ¹H NMR (DMSO-d₆, 400 MHz): δ ppm 7.76 (d, J=7.8 Hz, 2H), 7.32 (s, 1H), 7.10 (t, J=7.8 Hz, 1H), 6.69-6.75 (m, 1H), 5.72-5.83 (m, 1H), 4.19-4.30 (m, 2H), 3.85 (s, 3H), 2.61-2.75 (m, 4H), 2.11 (s, 3H). MS obsd. (ESI⁺) [(M+H)⁺]:391.0.

### Example 10

### 3-[5-chloro-2-(8-chloro-4-oxo-chroman-2-yl)-4-methoxy-phenoxy]propanoic Acid

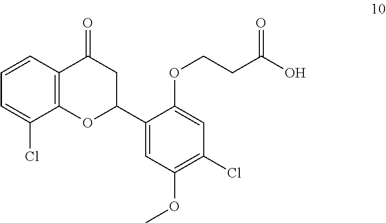 [SMILES: COc1cc(C2CC(=O)c3cccc(Cl)c3O2)c(OCCC(=O)O)cc1Cl]

### Step 1: Preparation of 4-chloro-2-hydroxy-5-methoxy-benzaldehyde

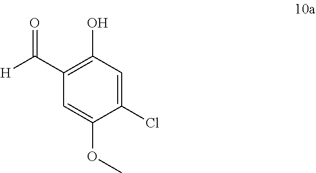

Compound 10a was prepared in analogy to the procedure described for the preparation of compound 7a by using 3-bromo-4-methoxy-phenol as the starting material instead of 3-bromo-4-methyl-phenol.

### Step 2: Preparation of 3-[5-chloro-2-(8-chloro-4-oxo-chroman-2-yl)-4-methoxy-phenoxy]propanoic Acid

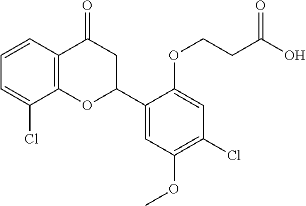 [SMILES: COc1cc(C2CC(=O)c3cccc(Cl)c3O2)c(OCCC(=O)O)cc1Cl]

Example 10 was prepared in analogy to the procedure described for the preparation of example 1 by using 4-chloro-2-hydroxy-5-methoxy-benzaldehyde as the starting material instead of 2-hydroxy-4-(trifluoromethyl)benzaldehyde in Step 1. ¹H NMR (DMSO-d₆, 400 MHz): δ ppm 12.37 (s, 1H), 7.78 (ddd, J=7.7, 5.8, 1.6 Hz, 2H), 7.39 (s, 1H), 7.29 (s, 1H), 7.13 (t, J=7.8 Hz, 1H), 5.87 (dd, J=13.6, 2.7 Hz, 1H), 4.05-4.31 (m, 2H), 3.84 (s, 3H), 3.34 (s, 1H), 2.79 (dd, J=16.9, 2.8 Hz, 1H), 2.66 (d, J=5.9 Hz, 2H). MS obsd. (ESI⁺) [(M+H)⁺]:411.0.

### Example 11

### 3-[2-(8-chloro-4-oxo-chroman-2-yl)-4-methoxy-5-(trifluoromethyl)phenoxy]propanoic Acid

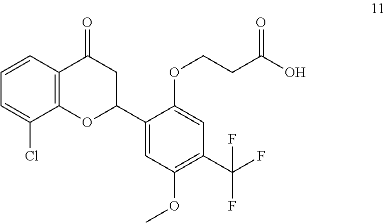 [SMILES: COc1cc(C2CC(=O)c3cccc(Cl)c3O2)c(OCCC(=O)O)cc1C(F)(F)F]

### Step 1: Preparation of 2-hydroxy-5-methoxy-4-(trifluoromethyl)benzaldehyde

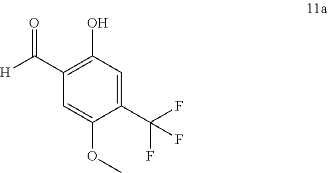

Compound 11a was prepared in analogy to the procedure described for the preparation of compound 7a by using 4-methoxy-3-(trifluoromethyl)phenol as the starting material instead of 3-bromo-4-methyl-phenol.

### Step 2: Preparation of 3-[2-(8-chloro-4-oxo-chroman-2-yl)-4-methoxy (trifluoromethyl)phenoxy]propanoic Acid

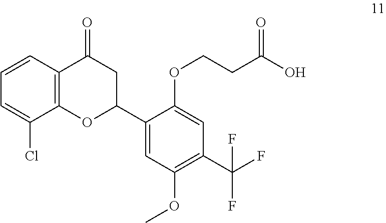 [SMILES: COc1cc(C2CC(=O)c3cccc(Cl)c3O2)c(OCCC(=O)O)cc1C(F)(F)F]

Example 11 was prepared in analogy to the procedure described for the preparation of example 1 by using 2-hydroxy-5-methoxy-4-(trifluoromethyl)benzaldehyde as the starting material instead of 2-hydroxy-4-(trifluoromethyl)benzaldehyde in Step 1. ¹H NMR (DMSO-d₆, 400 MHz): δ ppm 12.37 (s, 1H), 7.68-7.85 (m, 2H), 7.56 (s, 1H), 7.36 (s, 1H), 7.18-7.10 (m, 1H), 5.77-5.86 (m, 1H), 4.17-4.33 (m, 2H), 3.23 (dd, J=16.9, 13.5 Hz, 1H), 2.78 (dd, J=16.9, 2.8 Hz, 1H), 2.60-2.72 (m, 2H), 2.33 (s, 3H). MS obsd. (ESI⁺) [(M+H)⁺]:445.0.

### Example 12

### 3-[[6-(8-chloro-4-oxo-chroman-2-yl)-1,3-benzodioxol-5-yl]oxy]propanoic Acid

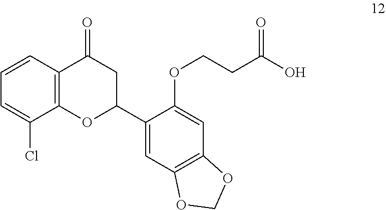 [SMILES: O=C(O)CCOc1cc2c(cc1C1CC(=O)c3cccc(Cl)c3O1)OCO2]

Example 12 was prepared in analogy to the procedure described for the preparation of example 1 by using 6-hydroxy-1,3-benzodioxole-5-carbaldehyde as the starting material instead of 2-hydroxy-4-(trifluoromethyl)benzaldehyde in Step 1. ¹H NMR (DMSO-d₆, 400 MHz): δ ppm 12.47 (s, 1H), 7.76 (d, J=7.7 Hz, 2H), 7.15 (s, 1H), 7.11 (t, J=7.8 Hz, 1H), 6.92 (s, 1H), 6.03 (d, J=4.8 Hz, 2H), 5.82 (dd, J=13.7, 2.4 Hz, 1H), 4.16 (dd, J=11.7, 6.0 Hz, 2H), 3.27 (s, 1H), 2.69 (dd, J=16.8, 2.5 Hz, 1H), 2.62 (t, J=5.9 Hz, 2H). MS obsd. (ESI⁺) [(M+H)⁺]:391.0.

### Example 13

### 3-[2-(8-chloro-4-oxo-chroman-2-yl)-5-(trifluoromethoxy)phenoxy]propanoic Acid

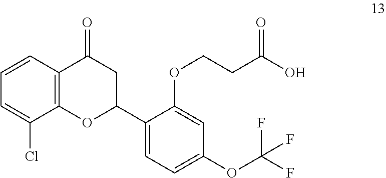 [SMILES: O=C(O)CCOc1cc(OC(F)(F)F)ccc1C1CC(=O)c2cccc(Cl)c2O1]

Example 13 was prepared in analogy to the procedure described for the preparation of example 1 by using 2-hydroxy-4-(trifluoromethoxy)benzaldehyde as the starting material instead of 2-hydroxy-4-(trifluoromethyl)benzaldehyde in Step 1. ¹H NMR (DMSO-d₆, 400 MHz): δ ppm 12.45 (s, 1H), 7.78 (dd, J=7.4, 6.1 Hz, 2H), 7.70 (d, J=8.5 Hz, 1H), 7.08-7.19 (m, 3H), 5.86 (dd, J=13.3, 2.5 Hz, 1H), 4.27 (ddd, J=15.4, 9.7, 3.8 Hz, 2H), 3.18-3.24 (m, 1H), 2.85 (dd, J=16.9, 2.7 Hz, 1H), 2.70 (t, J=5.8 Hz, 2H). MS obsd. (ESI⁺) [(M+H)⁺]:431.0.

### Example 14

### 3-[5-chloro-2-(8-chloro-4-oxo-chroman-2-yl)-4-methyl-phenoxy]propanoic Acid

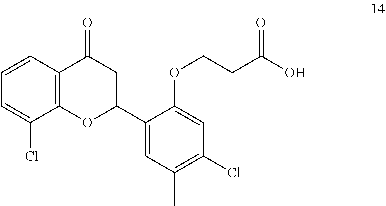 [SMILES: Cc1cc(C2CC(=O)c3cccc(Cl)c3O2)c(OCCC(=O)O)cc1Cl]

### Step 1: Preparation of 4-chloro-2-hydroxy-5-methyl-benzaldehyde

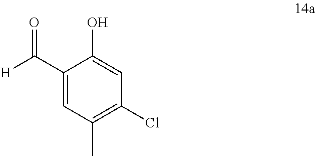

Compound 14a was prepared in analogy to the procedure described for the preparation of compound 7a by using 3-chloro-4-methyl-phenol as the starting material instead of 3-bromo methyl-phenol.

### Step 2: Preparation of 3-[5-chloro-2-(8-chloro-4-oxo-chroman-2-yl)-4-methyl-phenoxy]propanoic Acid

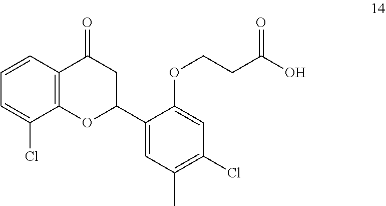 [SMILES: Cc1cc(C2CC(=O)c3cccc(Cl)c3O2)c(OCCC(=O)O)cc1Cl]

Example 14 was prepared in analogy to the procedure described for the preparation of example 1 by using 4-chloro-2-hydroxy-5-methyl-benzaldehyde as the starting material instead of 2-hydroxy-4-(trifluoromethyl)benzaldehyde in Step 1. ¹H NMR (DMSO-d₆, 400 MHz): δ ppm 7.76-7.79 (m, 2H), 7.55 (s, 1H), 7.22 (s, 1H), 7.12 (t, J=7.8 Hz, 1H), 5.81 (dd, J=13.5, 2.3 Hz, 1H), 4.17-4.28 (m, 2H), 3.20-3.27 (m, 1H), 2.78 (dd, J=16.9, 2.6 Hz, 1H), 2.67 (t, J=5.8 Hz, 2H), 2.31 (s, 3H). MS obsd. (ESI⁺) [(M+H)⁺]:395.0.

### Example 15

### 3-[4-bromo-2-(8-chloro-4-oxo-chroman-2-yl)-5-(trifluoromethoxy)phenoxy]propanoic Acid

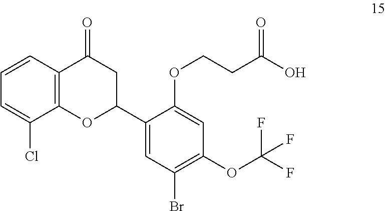 [SMILES: O=C(O)CCOc1cc(OC(F)(F)F)c(Br)cc1C1CC(=O)c2cccc(Cl)c2O1]

### Step 1: Preparation of 5-bromo-2-hydroxy-4-(trifluoromethoxy)benzaldehyde

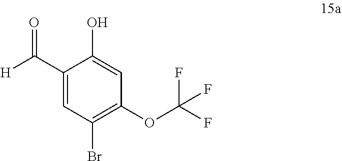 [SMILES: O=Cc1cc(Br)c(OC(F)(F)F)cc1O]

Compound 15a was prepared in analogy to the procedure described for the preparation of compound 7a by using 4-bromo-3-(trifluoromethoxy)phenol as the starting material instead of 3-bromo-4-methyl-phenol.

### Step 2: Preparation of 3-[4-bromo-2-(8-chloro-4-oxo-chroman-2-yl)-5-(trifluoromethoxy)phenoxy]propanoic Acid

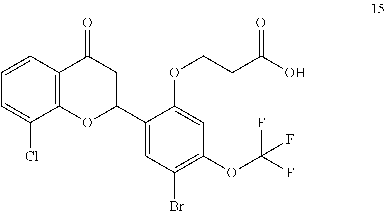 [SMILES: O=C(O)CCOc1cc(OC(F)(F)F)c(Br)cc1C1CC(=O)c2cccc(Cl)c2O1]

Example 15 was prepared in analogy to the procedure described for the preparation of example 1 by using 5-bromo-2-hydroxy-4-(trifluoromethoxy)benzaldehyde as the starting material instead of 2-hydroxy-4-(trifluoromethyl)benzaldehyde in Step 1. ¹H NMR (DMSO-d₆, 400 MHz): δ ppm 12.45 (s, 1H), 7.73-7.86 (m, 2H), 7.69 (s, 1H), 7.60 (s, 1H), 7.14 (t, J=7.8 Hz, 1H), 5.86 (dd, J=13.2, 2.4 Hz, 1H), 4.22-4.38(m, 2H), 3.14-3.24 (m, 1H), 2.88 (dd, J=16.9, 2.7 Hz, 1H), 2.71 (t, J=5.7 Hz, 2H). MS obsd. (ESI⁺) [(M+H)⁺]:509.0.

### Example 16

### 3-[5-bromo-2-(8-chloro-4-oxo-chroman-2-yl)phenoxy]propanoic Acid

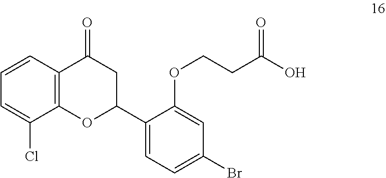 [SMILES: O=C(O)CCOc1cc(Br)ccc1C1CC(=O)c2cccc(Cl)c2O1]

Example 16 was prepared in analogy to the procedure described for the preparation of example 1 by using 4-bromo-2-hydroxy-benzaldehyde as the starting material instead of 2-hydroxy-4-(trifluoromethyl)benzaldehyde in Step 1. MS obsd. (ESI⁺) [(M+H)⁺]:425.0.

### Example 16-A and Example 16-B

### 3-[5-bromo-2-[(2S)-8-chloro-4-oxo-chroman-2-yl]phenoxy]propanoic Acid and 3-[5-bromo-2-[(2R)-8-chloro-4-oxo-chroman-2-yl]phenoxy]propanoic Acid

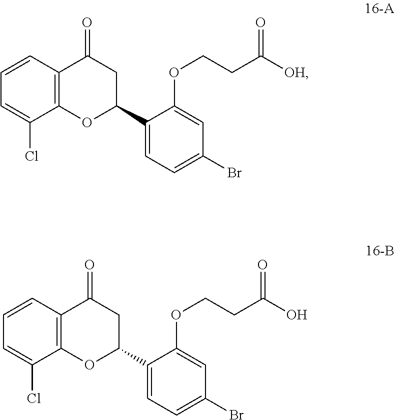 [SMILES: O=C(O)CCOc1cc(Br)ccc1[C@@H]1CC(=O)c2cccc(Cl)c2O1 ; O=C(O)CCOc1cc(Br)ccc1[C@H]1CC(=O)c2cccc(Cl)c2O1]

3-[5-bromo-2-(8-chloro-4-oxo-chroman-2-yl)phenoxy]propanoic Acid (250.0 mg, 0.590 mmol) was purified by chiral prep-HPLC (Instrument: Waters Acquity UPCCColumn: Daicel CHIRALPAK IG_3, 3.0*150 mm, 3 um Mobile Phase: CO₂/ETOH=85/15 Flow rate: 2.0 ml/min Column Temp: 37 degree) to give 2 enantiomers of 3-[5-bromo-2-(8-chloro-4-oxo-chroman-2-yl)phenoxy]propanoic acid with (+) and (−) configuration as white solid. The (+) configuration was characterized as Example 16-A (80 mg, 32.1% yield) and the (−) configuration was characterized as Example 16-B (84 mg, 33.6% yield).

Example 16-A: ¹H NMR (DMSO-d₆, 400 MHz): δ ppm 12.36 (s, 1H), 7.77 (td, J=7.9, 1.6 Hz, 2H), 7.52 (d, J=8.2 Hz, 1H), 7.35 (d, J=1.8 Hz, 1H), 7.30 (dd, J=8.2, 1.8 Hz, 1H), 7.12 (t, J=7.8 Hz, 1H), 5.82 (dd, J=13.2, 2.7 Hz, 1H), 4.19-4.34 (m, 2H), 3.19 (dd, J=16.9, 13.2 Hz, 1H), 2.83 (dd, J=16.9, 2.9 Hz, 1H), 2.66-2.74 (m, 2H). MS obsd. (ESI⁺) [(M+H)⁺]:424.9.

Example 16-B: ¹H NMR (DMSO-d₆, 400 MHz): δ ppm 12.36 (s, 1H), 7.77 (td, J=7.8, 1.6 Hz, 2H), 7.52 (d, J=8.2 Hz, 1H), 7.24-7.41 (m, 2H), 7.12 (t, J=7.8 Hz, 1H), 5.82 (dd, J=13.2, 2.7 Hz, 1H), 4.19-4.34 (m, 2H), 3.19 (dd, J=16.9, 13.2 Hz, 1H), 2.83 (dd, J=16.9, 2.9 Hz, 1H), 2.70 (t, J=5.9 Hz, 2H). MS obsd. (ESI⁺) [(M+H)⁺]:424.9.

### Example 17

### 3-[2-(8-chloro-4-oxo-chroman-2-yl)-5-(trifluoromethyl)phenoxy]cyclobutanecarboxylic Acid

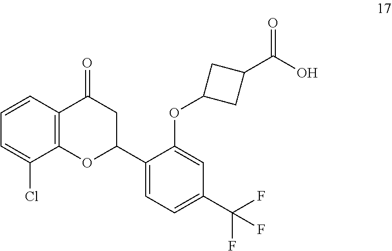 [SMILES: O=C1CC(c2ccc(C(F)(F)F)cc2OC2CC(C(=O)O)C2)Oc2c(Cl)cccc21]

### Step 1: Preparation of methyl 3-[2-formyl-5-(trifluoromethyl)phenoxy]cyclobutanecarboxylate

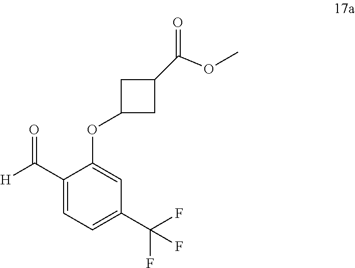 [SMILES: COC(=O)C1CC(Oc2cc(C(F)(F)F)ccc2C=O)C1]

To a mixture of 2-hydroxy-4-(trifluoromethyl)benzaldehyde (4.5 g, 23.7 mmol), methyl 3-chlorocyclobutane-1-carboxylate (5.28 g, 35.5 mmol) in DMF (10 mL) was added Cs₂CO₃ (15.4 g, 47.3 mmol) and the mixture was then stirred at 90° C. for overnight. After the reaction was completed, the mixture was diluted with water (30 mL) and the resulting mixture was extracted with EtOAc (50 mL) three times. The combined organic layer was washed with brine, dried over anhydrous Na₂SO₄ and concentrated in vacuo. The residue was purified by column chromatography on silica gel (eluting with PE:EtOAc=10:1) to give the methyl 3-[2-formyl-5-(trifluoromethyl)phenoxy]cyclobutanecarboxylate (5.0 g, 69.9% yield) as a yellow solid. MS obsd. (ESI⁺) [(M+H)⁺]:303.1.

### Step 2: Preparation of 3-[2-[(E)-3-(3-chloro-2-hydroxy-phenyl)-3-oxo-prop-1-enyl]-5-(trifluoromethyl)phenoxy]cyclobutanecarboxylic Acid

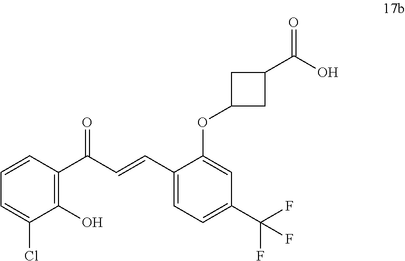 [SMILES: O=C(/C=C/c1ccc(C(F)(F)F)cc1OC1CC(C(=O)O)C1)c1cccc(Cl)c1O]

A mixture of KOH (6.57 g 99.3 mmol), methyl 3-[2-formyl-5-(trifluoromethyl)phenoxy]cyclobutanecarboxylate (5 g, 16.5 mmol) and 1-(3-chloro-2-hydroxyphenyl)ethan-1-one (2.81 g, 16.5 mmol) in EtOH (30 mL) was stirred at 80° C. overnight. After the reaction was completed, the reaction mixture was diluted with water (20 mL) adjusted to pH˜2 by addition of 1N HCl. The resulting suspension was extracted with EtOAc (50 mL) three times. The combined organic layer was washed with brine, dried over anhydrous Na₂SO₄ and concentrated in vacuo. The residue was purified by column chromatography on silica gel (eluting with PE:EtOAc=10:1 to 2:1) to give 3-[2-[(E)-3-(3-chloro-2-hydroxy-phenyl)-3-oxo-prop-1-enyl]-5-(trifluoromethyl)phenoxy]cyclobutanecarboxylic acid (2 g, 27.4% yield) as a yellow oil. MS obsd. (ESI⁺) [(M+H)⁺]:441.1.

### Step 3: Preparation of 3-[2-(8-chloro-4-oxo-chroman-2-yl)-5-(trifluoromethyl)phenoxy]cyclobutanecarboxylic Acid

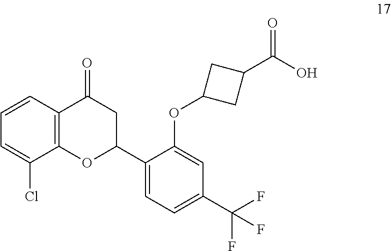 [SMILES: O=C1CC(c2ccc(C(F)(F)F)cc2OC2CC(C(=O)O)C2)Oc2c(Cl)cccc21]

To a solution of 3-[2-[(E)-3-(3-chloro-2-hydroxy-phenyl)-3-oxo-prop-1-enyl]-5-(trifluoromethyl)phenoxy]cyclobutanecarboxylic acid (1.5 g, 3.4 mmol) in MeOH (10 mL) and water (10 mL) was added pyridine (10 mL), the mixture was then stirred at 100° C. for 13 hours. After the reaction was completed, the mixture was diluted with water (30 mL) and adjusted to pH˜2 by addition of 1N HCl. The resulting mixture was extracted with EtOAc (50 mL) three times. The combined organic layer was washed with brine, dried over anhydrous Na₂SO₄ and concentrated in vacuo. The residue was then purified by preparative HPLC to give 3-[2-(8-chloro-4-oxo-chroman-2-yl)-5-(trifluoromethyl)phenoxy]cyclobutanecarboxylic acid (400 mg, 25.3% yield) as a light yellow solid. ¹H NMR (DMSO-d₆, 400 MHz): δ ppm 7.73-7.88 (m, 3H), 7.41-7.52 (m, 1H), 7.07-7.24 (m, 2H), 5.96-6.12 (m, 1H), 4.83-5.12 (m, 1H), 3.18-3.30 (m, 1H), 3.05-3.15 (m, 1H), 2.85-2.99 (m, 1H), 2.58-2.80 (m, 2H), 2.28-2.44 (m, 1H), 2.12-2.28 (m, 1H). MS obsd. (ESI⁺) [(M+H)⁺]:441.1.

### Example 18

### 3-[5-chloro-2-(8-chloro-4-oxo-chroman-2-yl)-4-methyl-phenoxy]cyclobutanecarboxylic Acid

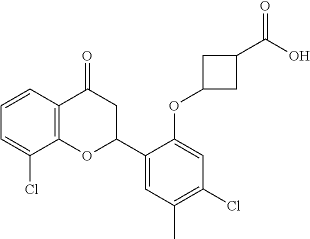 [SMILES: Cc1cc(C2CC(=O)c3cccc(Cl)c3O2)c(OC2CC(C(=O)O)C2)cc1Cl]

Example 18 was prepared in analogy to the procedure described for the preparation of example 17 by using 4-chloro-2-hydroxy-5-methylbenzaldehyde as the starting material instead of 2-hydroxy-4-(trifluoromethyl)benzaldehyde in Step 1. ¹H NMR (DMSO-d₆, 400 MHz): δ ppm 7.77 (d, J=7.83 Hz, 2H), 7.54-7.59 (m, 1H), 7.09-7.16 (m, 1H), 6.97-7.05 (m, 1H), 5.86-5.94 (m, 1H), 4.68-5.01 (m, 1H), 3.23-3.29 (m, 1H), 2.78-2.85 (m, 1H), 2.57-2.77 (m, 3H), 2.30 (s, 3H), 2.10-2.24 (m, 2H). MS obsd. (ESI⁺) [(M+H)⁺]:421.1.

### Example 19

### 3-[2-(8-chloro-4-oxo-chroman-2-yl)-5-(trifluoromethyl)phenoxy]-2,2-dimethyl-propanoic Acid

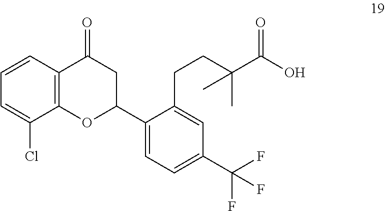 [SMILES: CC(C)(CCc1cc(C(F)(F)F)ccc1C1CC(=O)c2cccc(Cl)c2O1)C(=O)O]

Example 19 was prepared in analogy to the procedure described for the preparation of example 17 by using methyl 3-chloro-2,2-dimethyl-propanoate as the starting material instead of methyl 3-chlorocyclobutane-1-carboxylate in Step 1. ¹H NMR (DMSO-d₆, 400 MHz): δ ppm 12.36-12.55 (m, 1H), 7.70-7.86 (m, 3H), 7.38-7.53 (m, 2H), 7.10-7.21 (m, 1H), 5.82-5.94 (m, 1H), 4.03-4.23 (m, 2H), 3.17-3.27 (m, 1H), 2.83-2.94 (m, 1H), 1.21 (s, 6H). MS obsd. (ESI⁺) [(M+H)⁺]:443.1.

### Example 20

### 3-[2-[2-(8-chloro-4-oxo-chroman-2-yl)-5-(trifluoromethyl)phenoxy]ethoxy]cyclobutanecarboxylic Acid

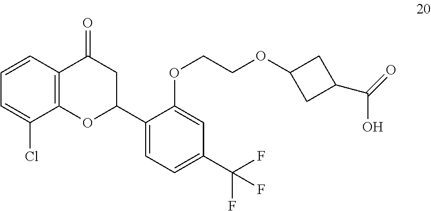 [SMILES: O=C1CC(c2ccc(C(F)(F)F)cc2OCCOC2CC(C(=O)O)C2)Oc2c(Cl)cccc21]

### Step 1: Preparation of 2-benzyloxyethoxy(trimethyl)silane

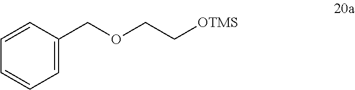

To a solution of 2-benzyloxyethanol (20.0 g, 131.4 mmol) and TEA (20.0 g, 197.1 mmol) in dichloromethane (200 mL) cooled at 0° C. was added trimethylsilyl chloride (17.1 g, 157.7 mmol) and the mixture was then stirred at 25° C. for 16 hours. After the reaction was completed, the mixture was concentrated in vacuo and the residue was purified by column chromatography on silica gel (eluted with PE:EtOAc=50:1 to 10:1) to give the 2-benzyloxyethoxy(trimethyl)silane (25.0 g, 84.9%) as a colorless oil.

### Step 2: Preparation of methyl 3-(2-benzyloxyethoxy)cyclobutanecarboxylate

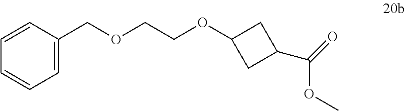 [SMILES: COC(=O)C1CC(OCCOCc2ccccc2)C1]

To a solution of 2-benzyloxyethoxy(trimethyl)silane (25.0 g, 111.4 mmol) and methyl 3-oxocyclobutanecarboxylate (CAS #: 4934-99-0, Cat. #: PB01390, from PharmaBlock (NanJing) R&D Co. Ltd, 15.0 g, 117.0 mmol) in dichloromethane (200 mL) was added trimethylsilyl trifluoromethanesulfonate (12.4 g, 55.7 mmol) dropwise at −78° C. After addition, the mixture was stirred at −78° C. for additional 1 hour, then to the resulting mixture was added triethylsilane (14.25 g, 122.57 mmol). After addition, the resulting mixture was warmed to room temperature and stirred for additional 1 hour. After the reaction was completed, the mixture was washed with saturated NH₄C1 solution, brine, dried over anhydrous sodium sulfate, and concentrated in vacuo. The residue was purified by column chromatography on silica gel (eluting with PE/EtOAc=100:1-50:1) to give methyl 3-(2-benzyloxyethoxy)cyclobutanecarboxylate (28 g, 95.1%) as a colorless oil. MS obsd. (ESI⁺) [(M+H)⁺]: 265.1.

### Step 3: Preparation of methyl 3-(2-hydroxyethoxy)cyclobutanecarboxylate

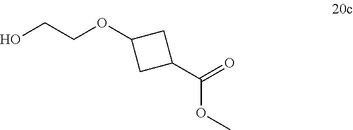

To a solution of methyl 3-(2-benzyloxyethoxy)cyclobutanecarboxylate (28.0 g, 105.9 mmol) in MeOH (300.0 mL) was added Pd(OH)₂ (wet) (1.48 g, 10.6 mmol) at room temperature and the mixture was then hydrogenated under H₂ atmosphere at room temperature overnight. After the reaction was completed, the reaction was filtered through silica gel pad and the filtrate was concentrated in vacuo to give 18 g crude methyl 3-(2-hydroxyethoxy)cyclobutanecarboxylate (18 g, 97.6%) as a colorless oil.

### Step 4: Preparation of methyl 3-[2-(p-tolylsulfonyloxy)ethoxy]cyclobutanecarboxylate

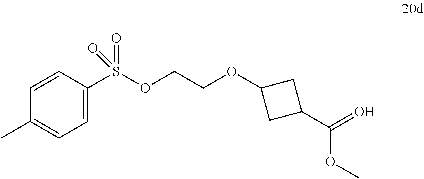

To a solution of methyl 3-(2-hydroxyethoxy)cyclobutanecarboxylate (5 g, 28.7 mmol) and DMAP (5.26 g, 43.1 mmol) in dichloromethane (80 mL) was added 4-methylbenzene-1-sulfonyl chloride (6.02 g, 31.6 mmol) at room temperature and the mixture was then stirred at room temperature overnight. After the reaction was completed, the mixture was washed with 1N HCl (25 mL), water (15 mL), saturated NaHCO₃ solution, brine and concentrated in vacuo to give the crude methyl 3-[2-(p-tolylsulfonyloxy)ethoxy]cyclobutanecarboxylate (8.1 g, 85.6%) as a colorless oil, which was used in next step directly without further purification. MS obsd. (ESI⁺) [(M+H)⁺]: 329.2.

### Step 5: Preparation of 3-[2-[2-(8-chloro-4-oxo-chroman-2-yl)-5-(trifluoromethyl)phenoxy]lethoxy]cyclobutanecarboxylic Acid

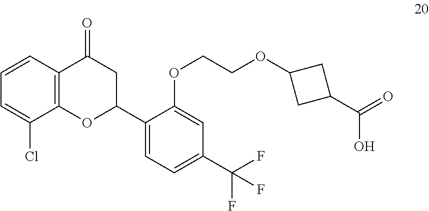 [SMILES: O=C1CC(c2ccc(C(F)(F)F)cc2OCCOC2CC(C(=O)O)C2)Oc2c(Cl)cccc21]

Example 20 was prepared in analogy to the procedure described for the preparation of example 17 by using methyl 3-[2-(p-tolylsulfonyloxy)ethoxy]cyclobutanecarboxylate as the starting material instead of methyl 3-chlorocyclobutane-1-carboxylate in Step 1. ¹H NMR (DMSO-d₆, 400 MHz): δ ppm 12.03-12.24 (m, 1H), 7.72-7.86 (m, 3H), 7.41-7.52 (m, 2H), 7.09-7.19 (m, 1H), 5.93-6.05 (m, 1H), 4.22-4.35 (m, 2H), 3.85-4.17 (m, 1H), 3.57-3.70 (m, 2H), 3.17-3.29 (m, 1H), 2.95-3.10 (m, 1H), 2.75-2.92 (m, 1H), 2.26-2.43 (m, 2H), 2.00-2.14 (m, 1H), 1.82-1.99 (m, 1H). MS obsd. (ESI⁺) [(M+H)⁺]:485.1.

### Example 21

### 2-[2-[2-(8-chloro-4-oxo-chroman-2-yl)-5-(trifluoromethyl)phenoxy]ethoxy]acetic Acid

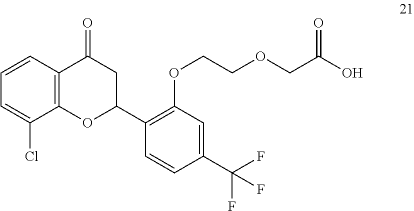 [SMILES: O=C(O)COCCOc1cc(C(F)(F)F)ccc1C1CC(=O)c2cccc(Cl)c2O1]

### Step 1: Preparation of methyl 2-(2-benzyloxyethoxy)acetate

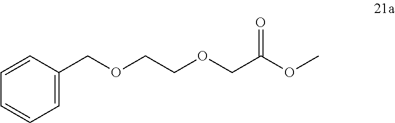 [SMILES: COC(=O)COCCOCc1ccccc1]

To a mixture of NaOH (10M, 300.0 mL), methyl 2-bromoacetate (23.5 g, 155.6 mmol) and tetrabutylammonium iodide (8.8 g, 24.06 mmol) in DCM (300 mL) was added 2-benzyloxyethanol (12.99 mL, 123.32 mmol) at 30° C. and the mixture was stirred at 30° C. for 72 hours. After the reaction was completed, the organic phase was separated out and the aquatic phase was extracted with DCM (150 mL) twice. The combined organic layer was washed with brine, dried over MgSO₄ and concentrated in vacuo. The residue was purified by column chromatography on silica gel (eluting with PE:EtOAc=3:1) to give methyl 2-(2-benzyloxyethoxy)acetate (21.3g, 78.9% yield) as a colorless liquid. MS obsd. (ESI⁺) [(M+Na)⁺]: 225.2.

### Step 2: Preparation of methyl 2-[2-(p-tolylsulfonyloxy)ethoxy]acetate

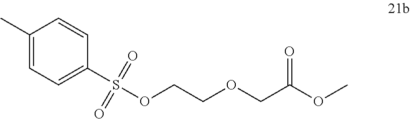

Compound 21b was prepared in analogy to the procedure described for the preparation of compound 20d by using methyl 2-(2-benzyloxyethoxy)acetate as the starting material instead of methyl 3-(2-benzyloxyethoxy)cyclobutanecarboxylate in Step 2. MS obsd. (ESI⁺) [(M+H)⁺]: 289.1

### Step 3: Preparation of 2-[2-[2-(8-chloro-4-oxo-chroman-2-yl)-5-(trifluoromethyl)phenoxy]ethoxy]acetic Acid

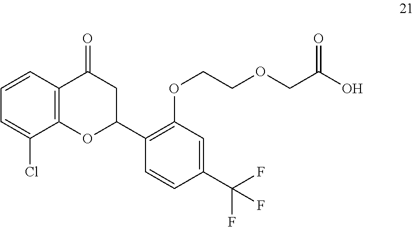 [SMILES: O=C(O)COCCOc1cc(C(F)(F)F)ccc1C1CC(=O)c2cccc(Cl)c2O1]

Example 21 was prepared in analogy to the procedure described for the preparation of example 17 by using methyl 2-[2-(p-tolylsulfonyloxy)ethoxy]acetate as the starting material instead of methyl 3-chlorocyclobutane-1-carboxylate in Step 1. ¹H NMR (DMSO-d₆, 400 MHz): δ ppm 7.75-7.86 (m, 3H), 7.40-7.52 (m, 2H), 7.05-7.20 (m, 1H), 5.94-6.07 (m, 1H), 4.24-4.38 (m, 2H), 4.04-4.09 (m, 2H), 3.76-3.88 (m, 2H), 3.12-3.27 (m, 1H), 2.94-3.04 (m, 1H). MS obsd. (ESI⁺) [(M+H)⁺]:445.1.

### Example 22

### 2-[3-[2-(8-chloro-4-oxo-chroman-2-yl)-5-methyl-phenoxy]propoxy]acetic Acid

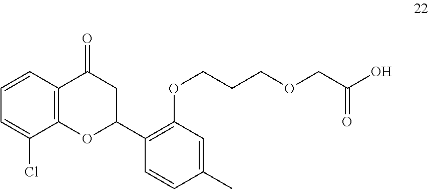 [SMILES: Cc1ccc(C2CC(=O)c3cccc(Cl)c3O2)c(OCCCOCC(=O)O)c1]

### Step 1: Preparation of tert-butyl 2-(3-(benzyloxy)propoxy)acetate

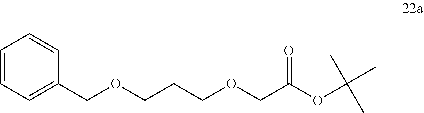 [SMILES: CC(C)(C)OC(=O)COCCCOCc1ccccc1]

To a mixture of NaOH (10M, 300.0 mL), tert-butyl 2-bromoacetate (23.5 g, 120.3 mmol) and tetrabutylammonium iodide (8.8 g, 24.06 mmol) in DCM (300 mL) was added 3-benzyloxypropan-1-ol (12.99 mL, 120.32 mmol) at 30° C. and the mixture was stirred for 72 hours. After the reaction was completed, the organic phase was separated out and the aquatic phase was extracted with DCM (150 mL) twice. The combined organic layer was washed with brine, dried over MgSO₄ and concentrated in vacuo. The residue was purified by column chromatography on silica gel (eluting with PE:EtOAc=3:1) to give tert-butyl 2-(3-(benzyloxy)propoxy)acetate (21.3 g, 63.3% yield) as a colorless liquid. MS obsd. (ESI⁺) [(M+Na)⁺]: 303.2.

### Step 2: Preparation of tert-butyl 2-[3-(p-tolylsulfonyloxy)propoxy]acetate

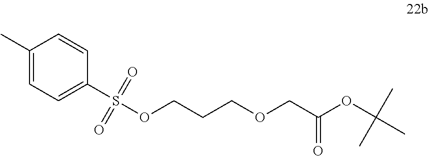 [SMILES: Cc1ccc(S(=O)(=O)OCCCOCC(=O)OC(C)(C)C)cc1]

Compound 22b was prepared in analogy to the procedure described for the preparation of compound 20d by using tert-butyl 2-(3-(benzyloxy)propoxy)acetate as the starting material instead of methyl 3-(2-benzyloxyethoxy)cyclobutanecarboxylate in Step 2. MS obsd. (ESI⁺) [(M+H)⁺]: 345.0.

### Step 3: Preparation of 2-[3-[2-(8-chloro-4-oxo-chroman-2-yl)-5-methyl-phenoxy]propoxy]acetic Acid

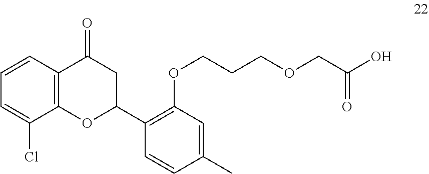 [SMILES: Cc1ccc(C2CC(=O)c3cccc(Cl)c3O2)c(OCCCOCC(=O)O)c1]

Example 22 was prepared in analogy to the procedure described for the preparation of example 17 by using 2-hydroxy-4-methyl-benzaldehyde and tert-butyl 2-[3-(p-tolylsulfonyloxy)propoxy]acetate as the starting materials instead of 2-hydroxy-4-(trifluoromethyl)benzaldehyde and methyl 3-chlorocyclobutane-1-carboxylate in Step 1. ¹H NMR (DMSO-d₆, 400 MHz): δ ppm 7.71-7.82 (m, 2H), 7.38-7.48 (m, 1H), 7.06-7.15 (m, 1H), 6.81-6.97 (m, 2H), 5.79-5.98 (m, 1H), 4.06-4.14 (m, 2H), 3.95 (s, 2H), 3.50-3.62 (m, 2H), 3.21-3.30 (m, 1H), 2.78-2.87 (m, 1H), 2.33 (s, 3H), 1.87-2.01 (m, 2H). MS obsd. (ESI⁺) [(M+H)⁺]:405.1.

### Example 23

### 2-[3-[2-(8-chloro-4-oxo-chroman-2-yl)-5-(trifluoromethyl)phenoxy]propoxy]acetic Acid

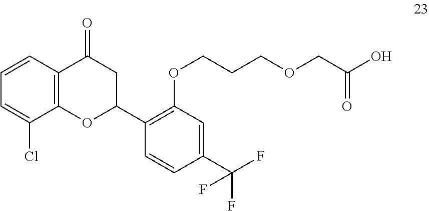 [SMILES: O=C(O)COCCCOc1cc(C(F)(F)F)ccc1C1CC(=O)c2cccc(Cl)c2O1]

Example 23 was prepared in analogy to the procedure described for the preparation of example 17 by using tert-butyl 2-[3-(p-tolylsulfonyloxy)propoxy]acetate as the starting material instead of methyl 3-chlorocyclobutane-1-carboxylate in Step 1. ¹H NMR (DMSO-d₆, 400 MHz): δ ppm 7.74-7.85 (m, 3H), 7.41-7.52 (m, 1H), 7.35-7.42 (m, 1H), 7.06-7.18 (m, 1H), 5.94-6.11 (m, 1H), 4.16-4.32 (m, 2H), 3.97 (s, 2H), 3.55-3.67 (m, 2H), 3.15-3.26 (m, 1H), 2.85-2.99 (m, 1H), 1.92-2.08 (m, 2H). MS obsd. (ESI⁺) [(M+H)⁺]:459.1.

### Example 24

### 2-[3-[2-(8-chloro-7-fluoro-4-oxo-chroman-2-yl)-5-(trifluoromethyl)phenoxy]propoxy]acetic Acid

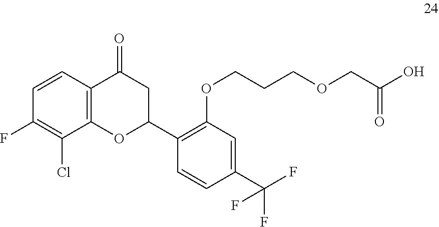 [SMILES: O=C(O)COCCCOc1cc(C(F)(F)F)ccc1C1CC(=O)c2ccc(F)c(Cl)c2O1]

Example 24 was prepared in analogy to the procedure described for the preparation of example 17 by using tert-butyl 2-[3-(p-tolylsulfonyloxy)propoxy]acetate as the starting material instead of methyl 3-chlorocyclobutane-1-carboxylate in Step 1 and using 1-(3-chloro-4-fluoro-2-hydroxy-phenyl)ethanone as the starting material instead of 1-(3-chloro-2-hydroxy-phenyl)ethanone in Step 2. ¹H NMR (DMSO-d₆, 400 MHz): δ ppm 12.59 (s, 1H), 7.85 (dd, J=8.8, 6.4 Hz, 1H), 7.80 (d, J=8.0 Hz, 1H), 7.46 (d, J=8.1 Hz, 1H), 7.39 (s, 1H), 7.21 (t, J=8.8 Hz, 1H), 6.10 (dd, J=13.2, 2.5 Hz, 1H), 4.16-4.28 (m, 2H), 3.96 (s, 2H), 3.60 (t, J=6.1 Hz, 2H), 3.24 (dd, J=16.8, 13.2 Hz, 1H), 2.92 (dd, J=16.9, 2.8 Hz, 1H), 1.98 (p, J=6.0 Hz, 2H). MS obsd. (ESI⁺) [(M+H)⁺]:477.0.

### Example 25

### 4-[2-(8-chloro-7-fluoro-4-oxo-chroman-2-yl)-5-(trifluoromethyl)phenoxy]butanoic Acid

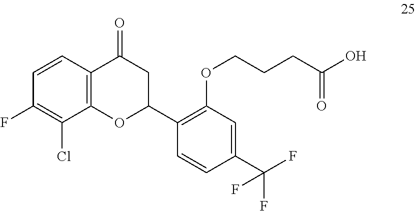 [SMILES: O=C(O)CCCOc1cc(C(F)(F)F)ccc1C1CC(=O)c2ccc(F)c(Cl)c2O1]

Example 25 was prepared in analogy to the procedure described for the preparation of example 17 by using methyl 4-bromobutyrate as the starting material instead of methyl 3-chlorocyclobutane-1-carboxylate in Step 1 and using 1-(3-chloro-4-fluoro-2-hydroxy-phenyl)ethanone as the starting material instead of 1-(3-chloro-2-hydroxy-phenyl)ethanone in Step 2. ¹H NMR (DMSO-d₆, 400 MHz): δ ppm 7.79-7.97 (m, 2H) 7.38 (d, J=7.9 Hz, 1H), 7.14 (d, J=11.6 Hz, 1H), 6.92 (t, J=8.5 Hz, 1H), 5.89 (d, J=12.2 Hz, 1H), 4.07-4.22 (m, 2H), 3.06 (dd, J=16.7, 2.3 Hz, 1H), 2.81 (dd, J=16.7, 13.5 Hz, 1H), 2.48-2.64 (m, 2H), 2.23-2.34 (m, 1H), 2.06-2.19 (m, 1H). MS obsd. (ESI⁺) [(M+H)⁺]:446.1.

### Example 26

### 2-[2-(8-chloro-7-fluoro-4-oxo-chroman-2-yl)-5-(trifluoromethyl)phenoxy]acetic Acid

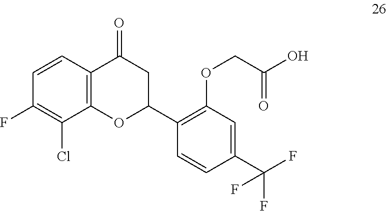 [SMILES: O=C(O)COc1cc(C(F)(F)F)ccc1C1CC(=O)c2ccc(F)c(Cl)c2O1]

Example 26 was prepared in analogy to the procedure described for the preparation of example 17 by using methyl bromoacetate as the starting material instead of methyl 3-chlorocyclobutane-1-carboxylate in Step 1 and using 1-(3-chloro-4-fluoro-2-hydroxy-phenyl)ethanone as the starting material instead of 1-(3-chloro-2-hydroxy-phenyl)ethanone in Step 2. ¹H NMR (DMSO-d₆, 400 MHz): δ ppm 13.18 (s, 1H), 7.80-7.91 (m, 2H), 7.50 (d, J=8.0 Hz, 1H), 7.39 (s, 1H), 7.22 (t, J=8.8 Hz, 1H), 6.10 (dd, J=12.9, 1.9 Hz, 1H), 4.96 (q, J=16.7 Hz, 2H), 3.25 (dd, J=16.9, 13.2 Hz, 1H), 3.03 (dd, J=16.9, 2.6 Hz, 1H). MS obsd. (ESI⁺) [(M+H)⁺]:419.0

### Example 27

### Methyl (2R)-3-[2-(8-chloro-4-oxo-chroman-2-yl)-5-(trifluoromethyl)phenoxy]-2-(ethylsulfonylamino)propanoate

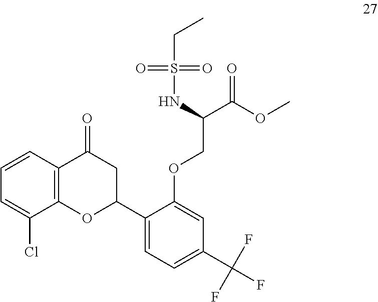 [SMILES: CCS(=O)(=O)N[C@H](COc1cc(C(F)(F)F)ccc1C1CC(=O)c2cccc(Cl)c2O1)C(=O)OC]

### Step 1: Preparation of 2-(methoxymethoxy)-4-(trifluoromethyl)benzaldehyde

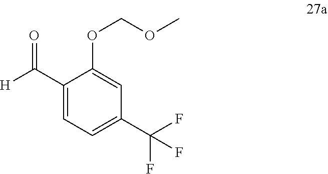 [SMILES: COCOc1cc(C(F)(F)F)ccc1C=O]

To a solution of 2-hydroxy-4-(trifluoromethyl)benzaldehyde (20.0 g, 105.2 mmol) in THF (100 mL) cooled at ice-water was added sodium hydride (3.53 g, 147.28 mmol) and the mixture was stirred at 0° C. for 20 mins. Then to the mixture was added bromomethyl methyl ether (18.6 g, 147.3 mmol) dropwise. After addition, the reaction mixture was stirred at r.t. for 1 hour. After the reaction was completed, the reaction solution was quenched with water (10 mL) and concentrated in vacuo. The residue was purified by column chromatography on silica gel (eluting with PE:EtOAc=10:1) to give 2-(methoxymethoxy)-4-(trifluoromethyl)benzaldehyde (17.0 g, 69.0% yield) as a white solid. MS obsd. (ESI⁺) [(M+Na)⁺]: 257.0.

### Step 2: Preparation of (E)-1-(3-chloro-2-hydroxy-phenyl)-3-[2-(methoxymethoxy)-4-(trifluoromethyl)phenyl]prop-2-en-1-one

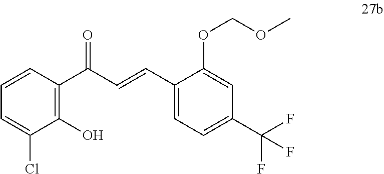 [SMILES: COCOc1cc(C(F)(F)F)ccc1/C=C/C(=O)c1cccc(Cl)c1O]

To a mixture of 1-(3-chloro-2-hydroxy-phenyl)ethanone (13.0 g, 76.23 mmol), 2-(methoxymethoxy)-4-(trifluoromethyl)benzaldehyde (17.0 g, 72.6 mmol) in EtOH (500 mL) was added KOH (24.4 g, 435.58 mmol) and the mixture was stirred at 35° C. for 16 hours. After the reaction was completed, the mixture was adjusted to pH˜4 by addition of HCl (1 M). The resulting suspension was filtered to give (E)-1-(3-chloro-2-hydroxy-phenyl)-3-[2-(methoxymethoxy)-4-(trifluoromethyl)phenyl]prop-2-en-1-one (27 g, 96.16% yield) as a light yellow solid. MS obsd. (ESI⁺)[(M+H)⁺]: 387.1.

### Step 3: Preparation of 8-chloro-2-[2-(methoxymethoxy)-4-(trifluoromethyl)phenyl]chroman-4-one

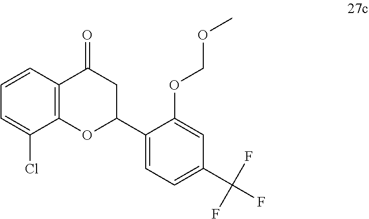 [SMILES: COCOc1cc(C(F)(F)F)ccc1C1CC(=O)c2cccc(Cl)c2O1]

To a solution of (E)-1-(3-chloro-2-hydroxy-phenyl)-3-[2-(methoxymethoxy) (trifluoromethyl)phenyl]prop-2-en-1-one (7.6 g, 19.65 mmol) in the mixed solvent of methanol (30 mL) and water (30 mL) was added pyridine (30.4 mL). The mixture was then stirred at 100° C. for 2 hours. After the reaction was completed, the mixture was concentrated in vacuo and the residue was purified by column chromatography on silica gel (eluting with PE:EtOAc=8:1) to give 8-chloro-2-[2-(methoxymethoxy)-4-(trifluoromethyl)phenyl]chroman-4-one (3.5 g, 46.05% yield) as a white solid. MS obsd. (ESI⁺)[(M+H)⁺]: 387.0.

### Step 4: Preparation of 8-chloro-2-[2-hydroxy-4-(trifluoromethyl)phenyl]chroman-4-one

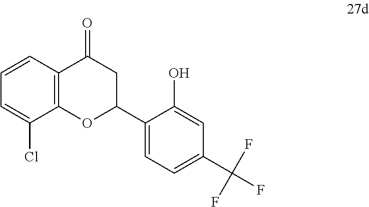 [SMILES: O=C1CC(c2ccc(C(F)(F)F)cc2O)Oc2c(Cl)cccc21]

To a solution of 8-chloro-2-[2-(methoxymethoxy)-4-(trifluoromethyl)phenyl]chroman-4-one (3.5 g, 9.05 mmol) in DCM (20 mL) was added TFA (5.58 mL, 72.4 mmol) and the mixture was stirred at r.t. for 2 hours. After the reaction was completed, the mixture was concentrated in vacuo to give the crude of 8-chloro-2-[2-hydroxy-4-(trifluoromethyl)phenyl]chroman-4-one (2.8 g, 90.28% yield) as a brown solid, which was used in the next step directly without further purification. MS obsd. (ESI⁺)[(M+H)⁺]: 343.0.

### Step 5: Preparation of methyl (2R)-2-amino-3-[2-(8-chloro-4-oxo-chroman-2-yl)-5-(trifluoromethyl) phenoxy]propanoate

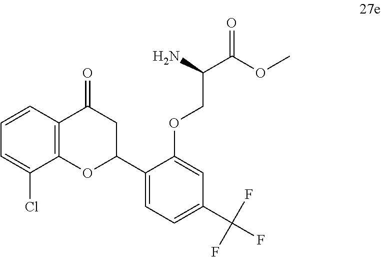 [SMILES: COC(=O)[C@H](N)COc1cc(C(F)(F)F)ccc1C1CC(=O)c2cccc(Cl)c2O1]

To a solution of 8-chloro-2-[2-hydroxy-4-(trifluoromethyl)phenyl]chroman-4-one (948.18 mg, 2.77 mmol), methyl (2R)-3-hydroxy-2-(tritylamino)propanoate (1000.0 mg, 2.77 mmol) and triphenylphosphine (1088.55 mg, 4.15 mmol) in THF (25 mL) was added DIAD (839.21 mg, 4.15 mmol) and the mixture was stirred at r.t. for 16 hours. The mixture was then diluted with water (30 mL) and extracted with EtOAc (30 mL) three times. The combined organic layer was washed with 1 N HCl (20 mL), brine (20 mL), dried over Na₂SO₄ and concentrated in vacuo.

The residue was dissolved in DCM (20 mL) and to the resulting solution was added TFA (3 mL). The mixture was stirred at r.t. for 2 hours. After the reaction was completed, the mixture was concentrated in vacuo and the residue was purified by column chromatography on silica gel (eluent with DCM:MeOH=20:1 to 10:1) to give methyl (2R)-2-amino-3-[2-(8-chloro-4-oxo-chroman-2-yl)-5-(trifluoromethyl)phenoxy]propanoate (1200 mg, 63.21% yield) as a light yellow solid. MS obsd. (ESI⁺)[(M+H)⁺]: 444.1.

### Step 6: Preparation of methyl (2R)-3-[2-(8-chloro-4-oxo-chroman-2-yl)-5-(trifluoromethyl)phenoxy]-2-(ethylsulfonylamino)propanoate

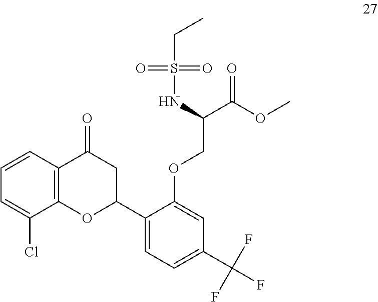 [SMILES: CCS(=O)(=O)N[C@H](COc1cc(C(F)(F)F)ccc1C1CC(=O)c2cccc(Cl)c2O1)C(=O)OC]

To a solution of methyl (2R)-2-amino-3-[2-(8-chloro-4-oxo-chroman-2-yl)-5-(trifluoromethyl)phenoxy]propanoate (100.0 mg, 0.230 mmol) and TEA (0.09 mL, 0.680 mmol) in DCM (4 mL) cooled at 0° C. was added ethanesulfonyl chloride (0.02 mL, 0.230 mmol) and the mixture was stirred at 0° C. for 1 hour. After the reaction was completed, the reaction mixture was quenched ice water (50 mL) and extracted with DCM (50 ml) three times. The combined organic layer was washed with brine (50 mL), dried with MgSO₄, filtered and concentrated in vacuo. The residue was purified by column chromatography on silica gel (eluting with PE:EtOAc=3:1 to 1:3) to give methyl (2R)-3-[2-(8-chloro-4-oxo-chroman-2-yl)-5-(trifluoromethyl)phenoxy]-2-(ethylsulfonylamino)propanoate (70 mg, 57.97% yield) as a light yellow solid. ¹H NMR (DMSO-d₆, 400 MHz): δ ppm 8.07 (s, 1H), 7.75-7.90 (m, 3H), 7.51 (br d, J=7.9 Hz, 1H), 7.36-7.47 (m, 1H), 7.16 (td, J=7.8, 5.4 Hz, 1H), 5.97-6.28 (m, 1H), 4.22-4.61 (m, 3H), 3.55-3.80 (m, 3H), 3.15-3.29 (m, 1H), 3.01-3.21 (m, 2H), 2.71-2.89 (m, 1H), 1.11-1.22 (m, 3H). MS obsd. (ESI⁺)[(M+H)⁺]: 536.1.

### Example 28-A and Example 28-B

### (2R)-3-[2-[(2S)-8-chloro-4-oxo-chroman-2-yl]-5-(trifluoromethyl)phenoxy]-2-(ethylsulfonylamino)propanoic Acid and (2R)-3-[2-[(2R)-8-chloro-4-oxo-chroman-2-yl]-5-(trifluoromethyl)phenoxy]-2-(ethylsulfonylamino)propanoic Acid

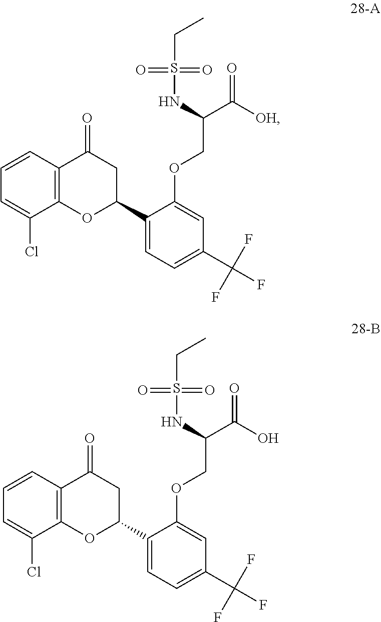

To a solution of methyl (2R)-3-[2-(8-chloro-4-oxo-chroman-2-yl)-5-(trifluoromethyl)phenoxy]-2-(ethylsulfonylamino)propanoate (90.0 mg, 0.170 mmol) in DCM (4 mL) was added BBr₃ (93.7 mg, 0.370 mmol) and the mixture was stirred at r.t. for 2 hours. After the reaction was completed, the reaction mixture was poured into ice water (30 mL) and extracted with DCM (30 mL) three times. The combined organic layer was washed with brine (30 mL), dried with MgSO₄ and concentrated in vacuo. The residue was purified by preparative HPLC to give two sets of diastereomers, one of which is characterized as Example 27-A (7.6 mg, 8.67% yield) and the other is Example 27-B (10.7 mg, 12.1% yield).

Example 27-A: ¹H NMR (DMSO-d₆, 400 MHz): δ ppm 7.82 (dd, J=19.1, 7.9 Hz, 3H), 7.41-7.53 (m, 2H), 7.15 (t, J=7.8 Hz, 1H), 6.54 (s, 1H), 6.04 (d, J=13.2 Hz, 1H), 4.38 (dd, J=26.2, 6.0 Hz, 3H), 3.09-3.20 (m, 1H), 2.98 (ddd, J=19.9, 16.0, 4.9 Hz, 3H), 1.15 (t, J=7.3 Hz, 3H). MS obsd. (ESI⁺) [(M+H)⁺]:522.0.

Example 27-B: ¹H NMR (DMSO-d₆, 400 MHz): δ ppm 7.61-7.95 (m, 3H), 7.33-7.57 (m, 2H), 7.15 (t, J=7.8 Hz, 1H), 6.54 (s, 1H), 6.25 (d, J=11.7 Hz, 1H), 4.51 (dd, J=9.5, 3.6 Hz, 1H), 4.18-4.39 (m, 2H), 2.94-3.13 (m, 2H), 2.82 (dd, J=16.8, 2.7 Hz, 1H), 1.02-1.24 (m, 3H). MS obsd. (ESI⁺) [(M+H)⁺]:522.1.

### Example 29

### (2S)-3-[2-(8-chloro-4-oxo-chroman-2-yl)-5-(trifluoromethyl)phenoxy]-2-(ethylsulfonylamino)propanoic Acid

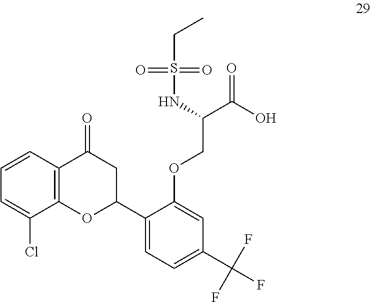 [SMILES: CCS(=O)(=O)N[C@@H](COc1cc(C(F)(F)F)ccc1C1CC(=O)c2cccc(Cl)c2O1)C(=O)O]

### Step 1: Preparation of methyl (2S)-2-amino-3-[2-(8-chloro-4-oxo-chroman-2-yl)-5-(trifluoromethyl) phenoxy]propanoate

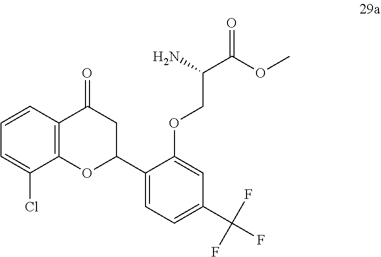 [SMILES: COC(=O)[C@@H](N)COc1cc(C(F)(F)F)ccc1C1CC(=O)c2cccc(Cl)c2O1]

Compound 29a was prepared in analogy to the procedure described for the preparation of compound 27e by using methyl (2S)-3-hydroxy-2-(tritylamino)propanoate as the starting material instead of methyl (2R)-3-hydroxy-2-(tritylamino)propanoate in Step 5. MS obsd. (ESI⁺) [(M+H)⁺]:442.1.

### Step 2: Preparation of methyl (2S)-3-[2-(8-chloro-4-oxo-chroman-2-yl)-5-(trifluoromethyl)phenoxy]-2-(ethylsulfonylamino)propanoate

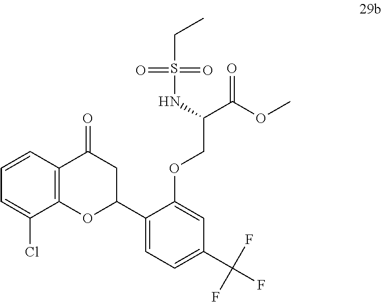 [SMILES: CCS(=O)(=O)N[C@@H](COc1cc(C(F)(F)F)ccc1C1CC(=O)c2cccc(Cl)c2O1)C(=O)OC]

Compound 29b was prepared in analogy to the procedure described for the preparation of example 27 by using methyl (2S)-2-amino-3-[2-(8-chloro-4-oxo-chroman-2-yl)-5-(trifluoromethyl) phenoxy]propanoate as the starting material instead of methyl (2R)-2-amino-3-[2-(8-chloro-4-oxo-chroman-2-yl)-5-(trifluoromethyl) phenoxy]propanoate in Step 6. MS obsd. (ESI⁺) [(M+H)⁺]:536.1.

### Step 3: Preparation of (25)-3-[2-(8-chloro-4-oxo-chroman-2-yl)-5-(trifluoromethyl)phenoxy]-2-(ethylsulfonylamino)propanoic Acid

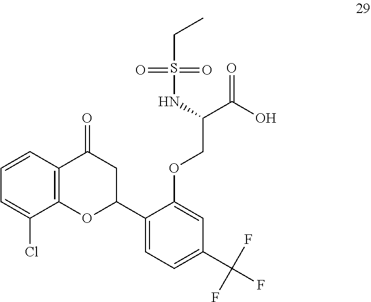 [SMILES: CCS(=O)(=O)N[C@@H](COc1cc(C(F)(F)F)ccc1C1CC(=O)c2cccc(Cl)c2O1)C(=O)O]

To a solution of methyl (2S)-3-[2-(8-chloro-4-oxo-chroman-2-yl)-5-(trifluoromethyl)phenoxy]-2-(ethylsulfonylamino)propanoate (100 mg, 0.18 mmol) in 1,2-dichloroethane (10 mL) was added hydroxy(trimethyl)stannane (67.5 mg, 0.7 mmol) and the mixture was then stirred at 80° C. for 6 hours. After the reaction was completed, the mixture was concentrated in vacuo and the residue was purified by preparative HPLC to give (2S)-3-[2-(8-chloro-4-oxo-chroman-2-yl)-5-(trifluoromethyl)phenoxy]-2-(ethylsulfonylamino)propanoic acid (37.0 mg, 37.5% yield) as a white solid. ¹H NMR (DMSO-d₆, 400 MHz): δ ppm 13.23 (s, 1H), 7.90-7.78 (m, 3H), 7.50 (d, J=8.0 Hz, 1H), 7.43 (d, J=20.4 Hz, 1H), 7.15 (td, J=7.8, 1.8 Hz, 1H), 6.16 (ddd, J=87.1, 13.0, 2.2 Hz, 1H), 4.54-4.21 (m, 3H), 3.22-3.08 (m, 1H), 3.07-2.99 (m, 2H), 2.88 (ddd, J=52.7, 16.9, 2.7 Hz, 1H), 1.16 (td, J=7.2, 4.6 Hz, 3H). MS obsd. (ESI⁺) [(M+H)⁺]:522.0.

### Example 30-A and Example 30-B

### (2R)-3-[2-[(2S)-8-chloro-4-oxo-chroman-2-yl]-5-(trifluoromethyl)phenoxy]-2-(sulfamoylamino)propanoic Acid and (2R)-3-[2-[(2R)-8-chloro-4-oxo-chroman-2-yl]-5-(trifluoromethyl)phenoxy]-2-(sulfamoylamino)propanoic Acid

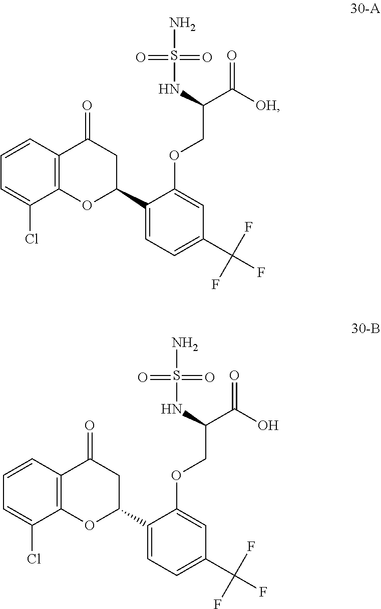 [SMILES: NS(=O)(=O)N[C@H](COc1cc(C(F)(F)F)ccc1[C@@H]1CC(=O)c2cccc(Cl)c2O1)C(=O)O ; NS(=O)(=O)N[C@H](COc1cc(C(F)(F)F)ccc1[C@H]1CC(=O)c2cccc(Cl)c2O1)C(=O)O]

### Step 1: Preparation of methyl (2R)-3-[2-(8-chloro-4-oxo-chroman-2-yl)-5-(trifluoromethyl)phenoxy]-2-(sulfamoylamino)propanoate

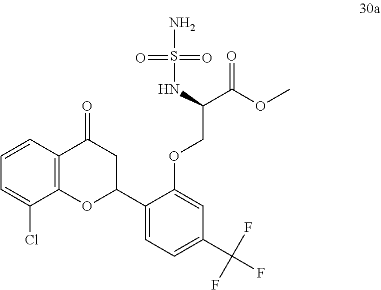 [SMILES: COC(=O)[C@@H](COc1cc(C(F)(F)F)ccc1C1CC(=O)c2cccc(Cl)c2O1)NS(N)(=O)=O]

Compound 30a was prepared in analogy to the procedure described for the preparation of example 27 by using sulfamoyl chloride as the starting material instead of ethanesulfonyl chloride in Step 6.

### Step 2: Preparation of (2R)-3-[2-[(2S)-8-chloro-4-oxo-chroman-2-yl]-5-(trifluoromethyl)phenoxy]-2-(sulfamoylamino)propanoic acid and (2R)-3-[2-[(2R)-8-chloro-4-oxo-chroman-2-yl]-5-(trifluoromethyl)phenoxy]-2-(sulfamoylamino)propanoic Acid

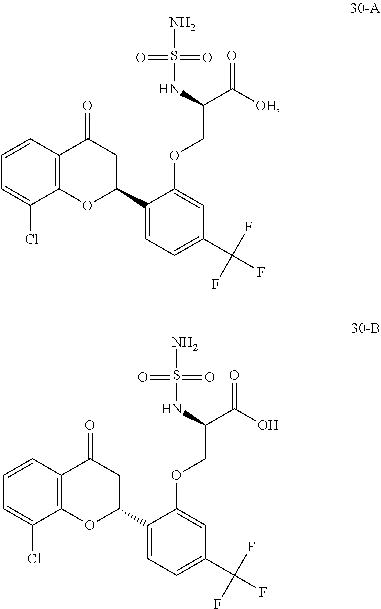 [SMILES: NS(=O)(=O)N[C@H](COc1cc(C(F)(F)F)ccc1[C@@H]1CC(=O)c2cccc(Cl)c2O1)C(=O)O ; NS(=O)(=O)N[C@H](COc1cc(C(F)(F)F)ccc1[C@H]1CC(=O)c2cccc(Cl)c2O1)C(=O)O]

Example 30-A and Example 30-B were prepared in analogy to the procedure described for the preparation of example 28-A and example 28-B by using methyl (2R)-3-[2-(8-chloro-4-oxo-chroman-2-yl)-5-(trifluoromethyl)phenoxy]-2-(sulfamoylamino)propanoate as the starting material instead of methyl (2R)-3-[2-(8-chloro-4-oxo-chroman-2-yl)-5-(trifluoromethyl)phenoxy]-2-(ethylsulfonylamino)propanoate.

Example 30-A: ¹H NMR (DMSO-d₆, 400 MHz): δ ppm 7.80 (ddd, J=7.9, 7.2, 4.7 Hz, 2H), 7.49 (d, J=8.3 Hz, 1H), 7.42 (s, 1H), 7.14 (t, J=7.8 Hz, 1H), 6.66 (s, 1H), 6.01 (d, J=12.8 Hz, 1H), 4.39 (s, 2H), 4.20 (s, 1H), 3.17-3.08 (m, 1H), 2.96 (d, J=16.5 Hz, 1H). MS obsd. (ESI⁺) [(M+H)^{+]:509.1.}

Example 30-B: ¹H NMR (DMSO-d₆, 400 MHz): δ ppm 7.79 (dd, J=7.5, 5.8 Hz, 2H), 7.47 (s, 1H), 7.39 (s, 1H), 7.14 (t, J=7.9 Hz, 1H), 6.62 (s, 2H), 6.16 (s, 1H), 4.47 (s, 1H), 4.28 (d, J=8.9 Hz, 1H), 3.10 — 2.99 (m, 1H), 2.92 (s, 1H). MS obsd. (ESI⁺) [(M+H)⁺]:509.1.

### Example 31-A and Example 31-B

### (2R)-3-[2-[(2S)-8-chloro-4-oxo-chroman-2-yl]-5-(trifluoromethyl)phenoxy]-2-(ethylsulfamoylamino)propanoic acid and (2R)-3-[2-[(2R)-8-chloro-4-oxo-chroman-2-yl]-5-(trifluoromethyl)phenoxy]-2-(ethylsulfamoylamino)propanoic Acid

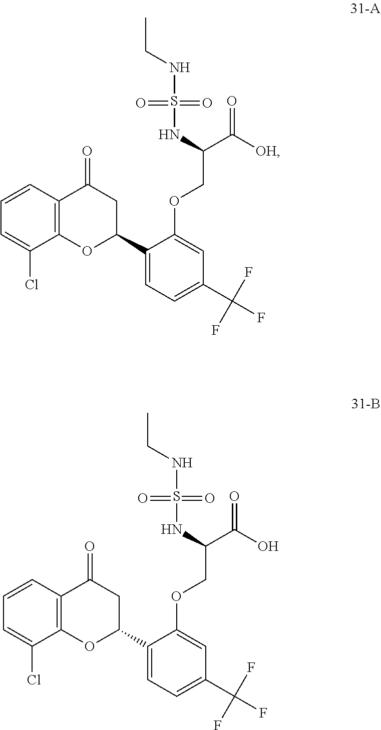 [SMILES: CCNS(=O)(=O)N[C@H](COc1cc(C(F)(F)F)ccc1[C@@H]1CC(=O)c2cccc(Cl)c2O1)C(=O)O ; CCNS(=O)(=O)N[C@H](COc1cc(C(F)(F)F)ccc1[C@H]1CC(=O)c2cccc(Cl)c2O1)C(=O)O]

### Step 1: Preparation of methyl (2R)-3-[2-(8-chloro-4-oxo-chroman-2-yl)-5-(trifluoromethyl)phenoxy]-2-(ethylsulfamoylamino)propanoate

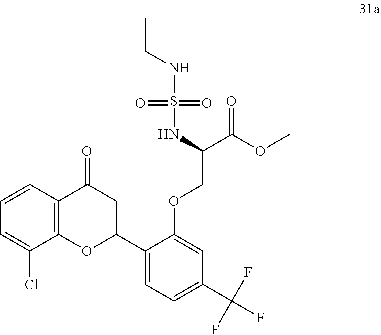 [SMILES: CCNS(=O)(=O)N[C@H](COc1cc(C(F)(F)F)ccc1C1CC(=O)c2cccc(Cl)c2O1)C(=O)OC]

Compound 31a was prepared in analogy to the procedure described for the preparation of example 27 by using N-ethylsulfamoyl chloride as the starting material instead of ethanesulfonyl chloride in Step 6.

### Step 2: Preparation of (2R)-3-[2-[(2S)-8-chloro-4-oxo-chroman-2-yl]-5-(trifluoromethyl)phenoxy]-2-(ethylsulfamoylamino)propanoic Acid and (2R)-3-[2-[(2R) chloro-4-oxo-chroman-2-yl]-5-(trifluoromethyl)phenoxy]-2-(ethylsulfamoylamino)propanoic Acid

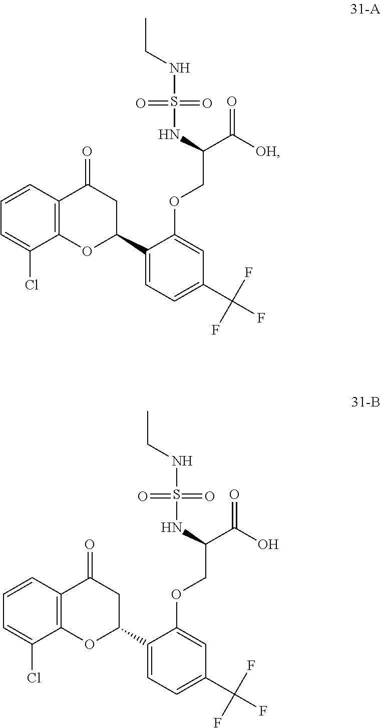 [SMILES: CCNS(=O)(=O)N[C@H](COc1cc(C(F)(F)F)ccc1[C@@H]1CC(=O)c2cccc(Cl)c2O1)C(=O)O ; CCNS(=O)(=O)N[C@H](COc1cc(C(F)(F)F)ccc1[C@H]1CC(=O)c2cccc(Cl)c2O1)C(=O)O]

Example 31-A and Example 31-B were prepared in analogy to the procedure described for the preparation of example 28-A and example 28-B by using methyl (2R)-3-[2-[(2S)-8-chloro-4-oxo-chroman-2-yl]-5-(trifluoromethyl)phenoxy]-2-(ethylsulfamoylamino)propanoate as the starting material instead of methyl (2R)-3-[2-(8-chloro-4-oxo-chroman-2-yl)-5-(trifluoromethyl)phenoxy]-2-(ethylsulfonylamino)propanoate.

Example 31-A: ¹H NMR (DMSO-d₆, 400 MHz): δ ppm 7.76-7.83 (m, 3H), 7.46 (d, J=8.1 Hz, 1H), 7.41 (s, 1H), 7.15 (s, 1H), 6.86 (br s, 1H), 5.97 (br d, J=12.8 Hz, 1H), 4.26-4.41 (m, 2H), 3.85 (br d, J=6.4 Hz, 1H), 3.14 (dd, J=16.8, 13.1 Hz, 1H), 2.92-3.01 (m, 1H), 2.82 (br d, J=6.5 Hz, 2H)), 0.97 (t, J=7.2 Hz, 3H). MS obsd. (ESI⁺) [(M+H)⁺]:537.0.

Example 31-B: ¹H NMR (DMSO-d₆, 400 MHz): δ ppm 7.75-7.85 (m, 3H), 7.48 (br d, J=7.9 Hz, 1H), 7.41 (s, 1H), 7.15 (s, 1H), 6.84 (br s, 1H), 6.19 (br d, J=12.1 Hz, 1H), 4.48 (br dd, J=9.4, 3.7 Hz, 1H), 4.26 (br dd, J=9.5, 2.9 Hz, 1H), 4.01 (br s, 1H), 3.06 (br dd, J=16.9, 13.2 Hz, 1H), 2.75-2.90 (m, 3H), 0.96 (t, J=7.2 Hz, 3H). MS obsd. (ESI⁺) [(M+H)⁺]:537.0.

### Example 32

### 8-chloro-7-fluoro-2-[2-(3-methoxypropoxy)-4-(trifluoromethyl)phenyl]chroman-4-one

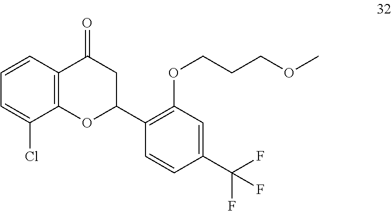 [SMILES: COCCCOc1cc(C(F)(F)F)ccc1C1CC(=O)c2cccc(Cl)c2O1]

### Step 1: Preparation of 2-(3-methoxypropoxy)-4-(trifluoromethyl)benzaldehyde

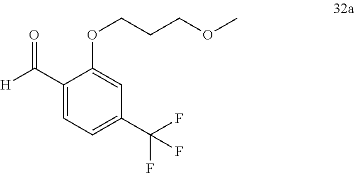 [SMILES: COCCCOc1cc(C(F)(F)F)ccc1C=O]

To a solution of 2-hydroxy-4-(trifluoromethyl)benzaldehyde (1000.0 mg, 5.26 mmol), 1-bromo-3-methoxypropane (1207.29 mg, 7.89 mmol) and KI (87.31 mg, 0.530 mmol) in DMF (54 mL) was added K₂CO₃ (2180.82 mg, 15.78 mmol) and the mixture was then stirred at 60° C. for 1 hour. After the reaction was completed, the mixture was diluted with water (50 mL) and the resulting solution was extracted with EtOAc (50 mL) three times. The combined organic layer was washed with brine, dried over Na₂SO₄ and concentrated in vacuo. The residue was purified by column chromatography on silica gel (eluting with PE:EtOAc=100:1 to 10:1) to give 2-(3-methoxypropoxy)-4-(trifluoromethyl)benzaldehyde (1200 mg, 487% yield) as a yellow solid. MS obsd. (ESI⁺)[(M+H)⁺]: 263.1.

### Step 2: Preparation of (E)-1-(3-chloro-4-fluoro-2-hydroxy-phenyl)-3-[2-(3-methoxypropoxy)-4-(trifluoromethyl)phenyl]prop-2-en-1-one

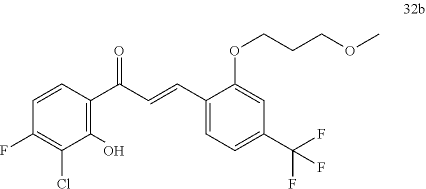 [SMILES: COCCCOc1cc(C(F)(F)F)ccc1/C=C/C(=O)c1ccc(F)c(Cl)c1O]

To a solution of 1-(3-chloro-4-fluoro-2-hydroxy-phenyl)ethanone (250.0 mg, 1.33 mmol) and 2-(3-methoxypropoxy)-4-(trifluoromethyl)benzaldehyde (347.6 mg, 1.33 mmol) in EtOH (15 mL) was added KOH (743.7 mg, 13.26 mmol) and the mixture was then stirred at 35° C. for 12 hours. After the reaction was completed, the reaction mixture was added water (20 mL) and the resulting mixture was adjusted to pH˜6 by addition of 1N HCl. The mixture was then filtered, the filter cake was collected and dried in vacuo to give (E)-1-(3-chloro-4-fluoro-2-hydroxy-phenyl)-3-[2-(3-methoxypropoxy)-4-(trifluoromethyl)phenyl]prop-2-en-1-one (450 mg, 78.43% yield) as a yellow solid. MS obsd. (ESI⁺)[(M+H)⁺]: 433.0.

### Step 3: Preparation of 8-chloro-7-fluoro-2-[2-(3-methoxypropoxy)-4-(trifluoromethyl)phenyl]chroman-4-one

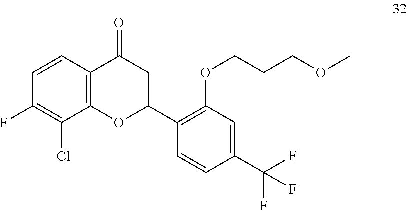 [SMILES: COCCCOc1cc(C(F)(F)F)ccc1C1CC(=O)c2ccc(F)c(Cl)c2O1]

To a solution of (E)-1-(3-chloro-4-fluoro-2-hydroxy-phenyl)-3-[2-(3-methoxypropoxy)-4-(trifluoromethyl)phenyl]prop-2-en-1-one (250.0 mg, 0.580 mmol) in water (6.97 mL) and methanol (6.97 mL) was added pyridine (2.4 mL, 29.62 mmol) and the mixture was stirred at 110° C. for 16 hours. After the reaction was completed, the mixture was concentrated in vacuo and the residue was purified preparative HPLC to give 8-chloro-7-fluoro-2-[2-(3-methoxypropoxy)-4-(trifluoromethyl)phenyl]chroman-4-one (28 mg, 11.13% yield) as a light yellow solid. ¹H NMR (DMSO-d₆, 400 MHz): δ ppm 7.80 (d, J=8.0 Hz, 1H), 7.86 (dd, J=8.7, 6.5 Hz, 1H), 7.46 (d, J=8.0 Hz, 1H), 7.40 (s, 1H), 7.22 (t, J=8.8 Hz, 1H), 6.09 (d, J=11.4 Hz, 1H), 4.19 (t, J=5.1 Hz, 2H), 3.46 (t, J=6.2 Hz, 2H), 3.26 (dd, J=16.9, 13.4 Hz, 1H), 3.21 (s, 3H), 2.91(dd, J=16.8, 2.5 Hz, 1H), 1.96 (p, J=6.0 Hz, 2H). MS obsd. (ESI⁺) [(M+H)⁺]:433.0.

### Example 33

### 8-chloro-7-fluoro-2-[2-(3-hydroxypropoxy)-4-(trifluoromethyl)phenyl]chroman-4-one

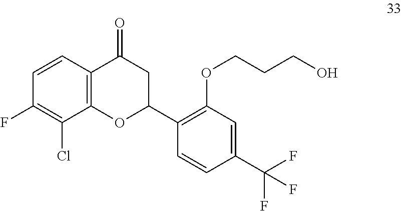 [SMILES: O=C1CC(c2ccc(C(F)(F)F)cc2OCCCO)Oc2c1ccc(F)c2Cl]

Example 33 was prepared in analogy to the procedure described for the preparation of example 32 by using 3-bromopropan-1-ol as the starting material instead of 1-bromo methoxypropane in Step 1. ¹H NMR (DMSO-d₆, 400 MHz): δ ppm 7.83 (dd, J=8.9, 6.4 Hz, 1H) 7.78 (d, J=8.0 Hz, 1H), 7.43 (d, J=8.0 Hz, 1H), 7.37 (s, 1H), 7.19 (t, J=8.8 Hz, 1H), 6.05 (dd, J=13.2, 2.5 Hz, 1H), 4.47 (s, 1H), 4.11-4.27 (m, 2H), 3.52 (t, J=6.2 Hz, 2H), 3.22 (dd, J=16.9, 13.3 Hz, 1H), 2.89 (dd, J=16.9, 2.8 Hz, 1H), 1.85 (t, J=6.1 Hz, 2H). MS obsd. (ESI⁺) [(M+H)⁺]:419.1.

### Example 34

### 8-chloro-7-fluoro-2-[2-(2-hydroxyethoxy)-4-(trifluoromethyl)phenyl]chroman-4-one

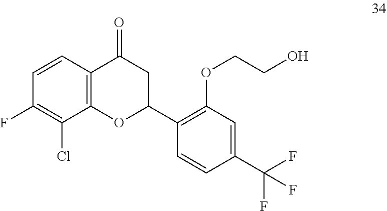 [SMILES: O=C1CC(c2ccc(C(F)(F)F)cc2OCCO)Oc2c1ccc(F)c2Cl]

Example 34 was prepared in analogy to the procedure described for the preparation of example 32 by using 2-bromoethanol as the starting material instead of 1-bromo-3-methoxypropane in Step 1. ¹H NMR (DMSO-d₆, 400 MHz): δ ppm 7.87 (dd, J=8.8, 6.5 Hz, 1H), 7.82 (d, J=7.9 Hz, 1H), 7.46 (d, J=8.0 Hz, 1H), 7.41 (s, 1H), 7.22 (t, J=8.8 Hz, 1H), 6.19 (dd, J=13.2, 2.4 Hz, 1H), 4.93 (t, J=5.8 Hz, 1H), 4.22-4.11 (m, 2H), 3.77-3.65 (m, 2H), 3.22 (dd, J=16.9, 13.3 Hz, 1H), 2.95 (dd, J=16.9, 2.8 Hz, 1H). MS obsd. (ESI⁺) [(M+H)⁺]:405.0.

### Example 35

### Ethyl 2-[2-[2-(8-chloro-4-oxo-chroman-2-yl)-5-(trifluoromethyl)phenoxy]ethylamino]-2-oxo-acetate

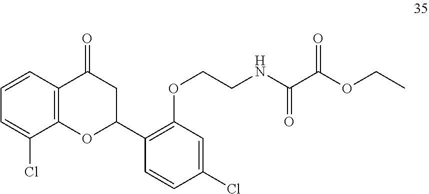 [SMILES: CCOC(=O)C(=O)NCCOc1cc(Cl)ccc1C1CC(=O)c2cccc(Cl)c2O1]

### Step 1: Preparation of tert-butyl N-[2-(5-chloro-2-formyl-phenoxy)ethyl]carbamate

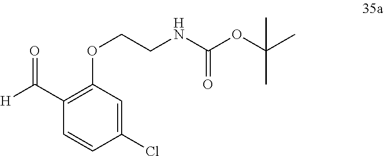 [SMILES: CC(C)(C)OC(=O)NCCOc1cc(Cl)ccc1C=O]

Compound 35a was prepared in analogy to the procedure described for the preparation of compound 32a by using tert-butyl N-(2-bromoethyl)carbamate and 4-chloro-2-hydroxy-benzaldehyde as the starting materials instead of 1-bromo-3-methoxypropane and 2-hydroxy-4-(trifluoromethyl)benzaldehyde in Step 1.

### Step 2: Preparation of tert-butyl N-[2-[5-chloro-2-(8-chloro-4-oxo-chroman-2-yl)phenoxy]ethyl]carbamate

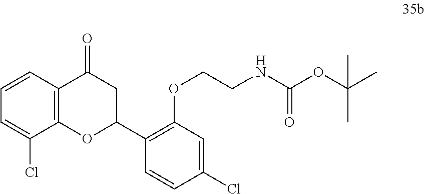 [SMILES: CC(C)(C)OC(=O)NCCOc1cc(Cl)ccc1C1CC(=O)c2cccc(Cl)c2O1]

Compound 35b was prepared in analogy to the procedure described for the preparation of example 32 by using tert-butyl N-[2-(5-chloro-2-formyl-phenoxy)ethyl]carbamate and 1-(3-chloro-2-hydroxy-phenyl)ethanone as the starting materials instead of 2-(3-methoxypropoxy)-4-(trifluoromethyl)benzaldehyde and 1-(3-chloro-4-fluoro-2-hydroxy-phenyl)ethanone in Step 2. MS obsd. (ESI⁺) [(M+Na)⁺]:474.0.

### Step 3: Preparation of 2-[2-(2-aminoethoxy)-4-chloro-phenyl]-8-chloro-chroman-4-one

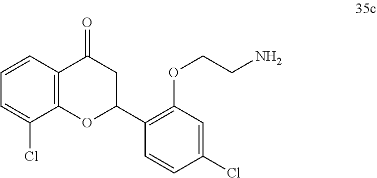 [SMILES: NCCOc1cc(Cl)ccc1C1CC(=O)c2cccc(Cl)c2O1]

To a solution of tert-butyl N-[2-[5-chloro-2-(8-chloro-4-oxo-chroman-2-yl)phenoxy]ethyl]carbamate (180.0 mg, 040 mmol) in DCM (10 mL) was added TFA (2 mL) and the mixture was then stirred at r.t. for 2 hours. After the reaction was completed, the mixture was concentrated in vacuo to give the crude of 2-[2-(2-aminoethoxy)-4-chloro-phenyl]-8-chloro-chroman-4-one (140.2 mg, 92.3% yield) as a yellow solid, which was used in the next step directly without further purification. MS obsd. (ESI⁺) [(M+H)⁺]: 352.1.

### Step 4: Preparation of ethyl 2-[2-[2-(8-chloro-4-oxo-chroman-2-yl)-5-(trifluoromethyl)phenoxy]ethylamino]-2-oxo-acetate

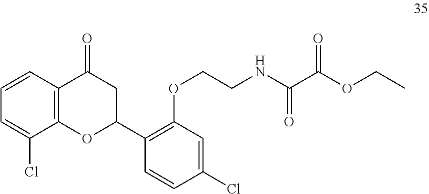 [SMILES: CCOC(=O)C(=O)NCCOc1cc(Cl)ccc1C1CC(=O)c2cccc(Cl)c2O1]

To a solution of 2-[2-(2-aminoethoxy)-4-chloro-phenyl]-8-chloro-chroman-4-one (140 mg, 0.39 mmol) in DCM (12 mL) cooled at 0° C. were added DIPEA (154.1 mg, 1.2 mmol) and ethyl oxalyl chloride (55.0 mg, 0.4 mmol) and the reaction was then stirred at r.t. for 1 hour. After the reaction was completed, the reaction was quenched with water (50 mL) and extracted with EtOAc (50 mL) three times. The combined organic layer was washed with brine, dried over Na₂SO₄ and concentrated in vacuo. The residue was purified by column chromatography on silica gel (eluting with PE:EtOAc=5:1) to give ethyl 2-[2-[2-(8-chloro-4-oxo-chroman-2-yl)-5-(trifluoromethyl)phenoxy]ethylamino]-2-oxo-acetate (130 mg, 72.3% yield) as a yellow solid. ¹H NMR (DMSO-d₆, 400 MHz): δ ppm 9.10 (t, J=5.7 Hz, 1H), 7.93-7.68 (m, 3H), 7.47 (d, J=8.1 Hz, 1H), 7.40 (s, 1H), 7.15 (t, J=7.8 Hz, 1H), 6.03 (dd, J=13.2, 2.5 Hz, 1H), 4.32-4.07 (m, 4H), 3.61-3.43 (m, 2H), 3.18 (dd, J=16.9, 13.3 Hz, 1H), 2.84 (dd, J=16.9, 2.8 Hz, 1H), 1.20 (t, J=7.1 Hz, 3H). MS obsd. (ESI⁺) [(M+H)⁺]:452.1.

### Example 36

### 2-[2-[5-chloro-2-(8-chloro-4-oxo-chroman-2-yl)phenoxy]ethylamino]-2-oxo-acetic Acid

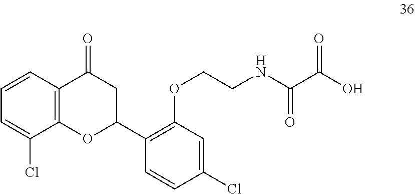 [SMILES: O=C(O)C(=O)NCCOc1cc(Cl)ccc1C1CC(=O)c2cccc(Cl)c2O1]

A solution of ethyl 2-[2-[2-(8-chloro-4-oxo-chroman-2-yl)-5-(trifluoromethyl)phenoxy]ethylamino]-2-oxo-acetate (130 mg 0.28 mmol) in 1 N HCl (2 mL) was stirred at 80° C. under microwave condition for 1.5 hours. After the reaction was completed, the mixture was concentrated in vacuo. The residue was purified by preparative HPLC to give 2-[2-[5-chloro-2-(8-chloro-4-oxo-chroman-2-yl)phenoxy]ethylamino]-2-oxo-acetic acid (7.8 mg, 6.2% yield) as a white solid. ¹H NMR (DMSO-d₆, 400 MHz): δ ppm 8.31-8.41 (m, 1H), 7.78 (d, J=7.8 Hz, 2H), 7.57 (d, J=8.3 Hz, 1H), 7.22 (d, J=1.7 Hz, 1H), 7.09-7.16 (m, 2H), 5.97-6.06 (m, 1H), 4.00-4.15 (m, 2H), 3.37-3.45 (m, 2H), 3.19 (dd, J=17.0, 13.1 Hz, 1H), 2.84 ppm (dd, J=16.9, 2.7 Hz, 1H). MS obsd. (ESI⁺) [(M+H)⁺]:424.1.

### Example 37

### cis-3-[5-bromo-2-(8-chloro-4-oxo-chroman-2-yl)phenoxy]cyclobutanecarboxylic Acid

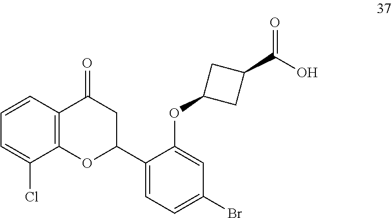 [SMILES: O=C1CC(c2ccc(Br)cc2O[C@H]2C[C@@H](C(=O)O)C2)Oc2c(Cl)cccc21]

### Step 1: Preparation of tert-butyl 3-(p-tolylsulfonyloxy)cyclobutanecarboxylate

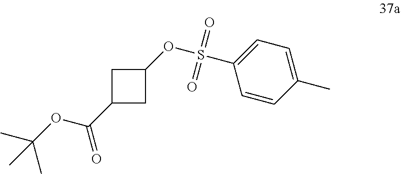 [SMILES: Cc1ccc(S(=O)(=O)OC2CC(C(=O)OC(C)(C)C)C2)cc1]

To a solution of tert-butyl 3-hydroxycyclobutanecarboxylate (1.00 g, 5.82 mmol) and TEA (1.17 g, 1.61 mL, 11.5 mmol) in dichloromethane (10 mL) was added methanesulfonyl chloride (1.92 g, 9.99 mmol) at 0° C. and the mixture was then stirred at room temperature overnight. The mixture was then diluted with dichloromethane (50 mL), the resulting solution was then washed with water (20 mL) twice, saturated NaHCO₃ (20 mL) twice, brine (20 mL), dried over anhydrous Na₂SO₄ and concentrated in vacuo to give the crude tert-butyl 3-(p-tolylsulfonyloxy)cyclobutanecarboxylate (1.9 g, 100%) as a colorless oil. MS obsd. (ESI⁺) [(M+H)⁺]: 326.2.

### Step 2: Preparation of cis-Cert-butyl 3-(5-bromo-2-formyl-phenoxy)cyclobutanecarboxylate

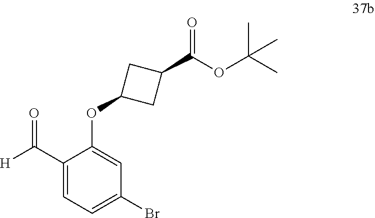 [SMILES: CC(C)(C)OC(=O)[C@H]1C[C@@H](Oc2cc(Br)ccc2C=O)C1]

To a solution of 4-bromo-2-hydroxybenzaldehyde (1.00 g, 4.97 mmol) and tert-butyl 3-(p-tolylsulfonyloxy)cyclobutanecarboxylate (1.62 g, 4.97 mmol) in DMF (26 mL) was added K₂CO₃ (1.38 g, 9.95 mmol) and the reaction mixture was stirred at 90° C. for 16 hours. After the reaction was completed, the reaction mixture was concentrated in vacuo and purified by preparative HPLC to give cis-tert-butyl 3-(5-bromo-2-formyl-phenoxy)cyclobutanecarboxylate (360 mg, 20.4% yield) as a white solid. MS obsd. (ESI⁺)[(M+Na)⁺]: 377.0.

### Step 3: Preparation of cis-3-[5-bromo-2-(8-chloro-4-oxo-chroman-2-yl)phenoxy]cyclobutanecarboxylic Acid

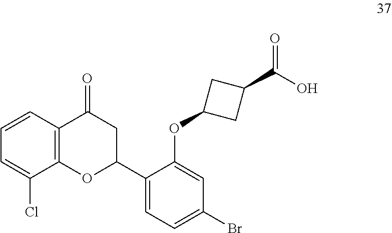 [SMILES: O=C1CC(c2ccc(Br)cc2O[C@H]2C[C@@H](C(=O)O)C2)Oc2c(Cl)cccc21]

Example 37 was prepared in analogy to the procedure described for the preparation of example 17 by using cis-tert-butyl 3-(5-bromo-2-formyl-phenoxy)cyclobutanecarboxylate as the starting material instead of methyl 3-[2-formyl-5-(trifluoromethyl)phenoxy]cyclobutanecarboxylate in Step 2. ¹H NMR (DMSO-d₆, 400 MHz): δ ppm 12.29 (s, 1H), 7.77 (d, J=7.7 Hz, 2H), 7.52 (d, J=8.2 Hz, 1H), 7.28 (d, J=8.0 Hz, 1H), 7.12 (t, J=7.6 Hz, 2H), 5.91 (d, J=11.5 Hz, 1H), 4.80 (dd, J=12.9, 6.2 Hz, 1H), 3.23 (dd, J=16.6, 13.5 Hz, 1H), 2.86 (dd, J=16.6, 1.9 Hz, 1H), 2.63-2.78 (m, 3H), 2.11-2.26 (m, 2H). MS obsd. (ESI⁺) [(M+H)⁺]: 451.0.

### Biological Examples

### BIO-Example 1: Engineered HepDES19 Primary Screen Assay

The assay was employed to screen for cccDNA inhibitors. HepDES19 is a cccDNA-producing cell line. In this cell line, HBeAg in the cell culture supernatant as surrogate marker, as HBeAg production depends on cccDNA level and activity. HepDES19 is an engineered cell line which contains a 1.1 unit length HBV genome, and pgRNA transcription from the transgene is controlled by Tetracycline (Tet). In the absence of Tet, pgRNA transcription will be induced, but HBV e antigen (HBeAg) could not be produced from this pgRNA due to very short leader sequence before the HBeAg start codon and the start codon is disrupted. Only after cccDNA is formed, the missing leader sequence and start codon mutation would be restored from the 3′-terminal redundancy of pgRNA, and then HBeAg could be synthesized. Therefore, HBeAg could be used as a surrogate marker for cccDNA (Zhou, T. et al., Antiviral Res. (2006), 72 (2), 116-124; Guo, H. et al., J. Virol. (2007), 81 (22), 12472-12484).

HepDES19 cells were seeded at 2×10⁶ cells per T150 flask and cultured with the culture medium (Dulbecco's Modified Eagle Medium: Nutrient Mixture F-12 [DMEM-F12, Gibco Cat. 11320-82], 10% Fetal Bovine Serum [FBS, Clontech Cat. 631101], 0.1 mM Non-Essential Amino Acids Solution [NEAA, Gibco Cat. 11140-050], 50 μg/mL Penicillin-Streptomycin [PS, Invitrogen Cat. 15140-163], 500 μg/mL Geneticin [G418, Invitrogen Cat. 10131-027]) containing 3 μg/mL Tet (Sigma, Cat. 87128) for 5 days. Cells were then seeded at 4×10⁶ cells per T150 in the same culture medium as described above in the absence of Tet for 8 days. Cells were then harvested and frozen at density of 2×10⁶ cells per mL. For compound testing, the frozen cells were thawed and seeded into 96-well plates at a density of 6×10⁴ cells per well. At 24 hours after seeding, half log serial dilutions of compounds made with Dimethyl sulfoxide (DMSO, Sigma, Cat. D2650) were further diluted with the same culture medium as described above before they were added to the cells to reach desired final compound concentrations and 1% DMSO concentration. Plates were then incubated at 37° C. for another 5 days before measurement of HBeAg level and cell viability. Intracellular HBeAg level were measured with enzyme-linked immunosorbent assay (ELISA) kit (Shanghai Kehua Diagnostic Medical Products Co., Ltd). Cell viability was assessed using Cell Counting Kit-8 (DonJindo, Cat. CK04-20). IC50 values were derived from the dose-response curve using 4 parameters logistic curve fit method.

The compounds of the present invention were tested for their capacity to inhibit extracellular HBeAg level as described herein. The compounds of this invention were found to have IC50 below 50 μM. Particular compounds of formula (I) were found to have IC50 below 5.0 μM. Results of HepDES19 primary screen assay are given in Table 1.

TABLE 1
Activity data in HepDES19 primary screen assay

| Example No. | IC₅₀ (μM) |
|---|---|
| 1 | 0.73 |
| 2 | 1.97 |
| 3 | 5.59 |
| 4 | 5.25 |
| 5 | 3.06 |
| 6 | 5.22 |
| 7 | 9.41 |
| 8 | 11.3 |
| 9 | 9.41 |
| 10 | 11.4 |
| 11 | 5.14 |
| 12 | 10.1 |
| 13 | 0.39 |
| 14 | 1.05 |
| 15 | 1.5 |
| 16-A | 2.43 |
| 16-B | 0.48 |
| 17 | 1.56 |
| 18 | 0.765 |
| 19 | 0.26 |
| 20 | 0.01 |
| 21 | 4.89 |
| 22 | 1.99 |
| 23 | 1.56 |
| 24 | 7.14 |
| 25 | 1.18 |
| 26 | 2.78 |
| 27 | 0.58 |
| 28 | 16.3 |
| 28 | 3.76 |
| 29 | 1.91 |
| 30-A | 3.37 |
| 30-B | 10.7 |
| 31-A | 3.3 |
| 31-B | 10.9 |
| 32 | 1.93 |
| 33 | 0.68 |
| 34 | 3.36 |
| 35 | 5.74 |
| 36 | 12.9 |
| 37 | 0.67 |

### BIO-Example 2: Cryopreserved Primary Human Hepatocytes (PHH) Assay

This assay is used to confirm the anti-HBV effect of the compounds in HBV PHH infection assay. Cryopreserved PHH (BioreclamationIVT, Lot YJM) was thawed at 37° C. and gently transferred into pre-warmed InVitroGRO HT medium (BioreclamationIVT, Cat. S03317).

The mixture was centrifuged at 70 relative centrifugal force (RCF) for 3 minutes at RT, and the supernatant was discarded. Pre-warmed InVitroGRO CP medium (BioreclamationIVT, Cat #S03316) was added to the cell pellet to gently re-suspend cells. The cells were seeded at the density of 5.8×10⁴ cells per well to collagen I coated 96-well plate (Gibco, Cat. A1142803) with the InVitroGRO CP medium. All plates were incubated at 37° C. with 5% CO₂ and 85% humidity.

At 20 hours after plating, the medium was changed to PHH culture medium (Dulbecco's Modified Eagle Medium (DMEM)/F12 (1:1) (Gibco, Cat. 11320-033), 10% fetal bovine serum (Gibco Cat. 10099141), 100 U/mL penicillin, 100 μg/mL streptomycin (Gibco, Cat. 151401-122), 5 ng/mL human epidermal growth factor (Invitrogen Cat. PHG0311L), 20 ng/mL dexamethasone (Sigma, Cat. D4902) and 250 ng/mL human recombinant insulin (Gibco, Cat. 12585-014)). And the cells were incubated at 37° C. with 5% CO₂ and 85% humidity for 4 hours. The medium was then changed to pre-warmed PHH culture medium containing 4% polyethylene glycol (PEG) MW8000 (Sigma, Cat. P1458-50 ML) and 1% DMSO (Sigma, Cat. D2650). 5.8×10⁶ genomic equivalents of HBV were added into the medium.

At 24 hours' post-infection, the cells were gently washed with PBS and refreshed with PHH culture medium supplemented with 1% DMSO, and 0.25 mg/mL Matrix gel (Corning, Cat. 356237) at 200 μL per well. All plates were immediately placed in at 37° C. CO₂ incubator.

24 hours later, serial dilutions of compounds made with DMSO were further diluted with the same culture medium (PHH culture medium supplemented with 1% DMSO and 0.25 mg/mL Matrix gel as described above) before they were added to the cells to reach desired final compound concentrations and 1% DMSO concentration. The medium containing the compounds were refreshed every three days.

At 9 days' post-compound treatment, extracellular HBsAg level were measured with Chemiluminescence Immuno Assay (CLIA) kit (Autobio, HBsAg Quantitative CLIA).

Extracellular HBV DNA was extracted by MagNA Pure 96 system (Roche) and then determined by quantitative PCR with the following primers and probe:

HBV-Forward Primer (SEQ ID NO: 1):
AAGAAAAACCCCGCCTGTAA (5′ to 3′);
HBV-Reverse Primer (SEQ ID NO: 2):
CCTGTTCTGACTACTGCCTCTCC (5′ to 3′)

HBV-Probe: 5′+tetramethylrhodamine+SEQ ID NO: 3+black hole quencher 2-3′, wherein SEQ ID NO: 3 is CCTGATGTGATGTTCTCCATGTTCAGC.

HBsAg IC₅₀ and HBV DNA IC₅₀ values were derived from the dose-response curve using 4 parameters logistic curve fit method. The compounds of formula (I) have HBsAg IC₅₀<20 μM, particularly <1 μM; and HBV DNA IC₅₀<50 μM. Results of Cryopreserved PHH assay are given in Table D2.

TABLE D2
HBsAg IC₅₀ data in Cryopreserved PHH assay

| Example No. | HBsAg IC₅₀ (μM) |
|---|---|
| 1 | 5.31 |
| 2 | 4.41 |
| 3 | 11.2 |
| 11 | 8.34 |
| 13 | 6.86 |

1. A compound of the formula (I), 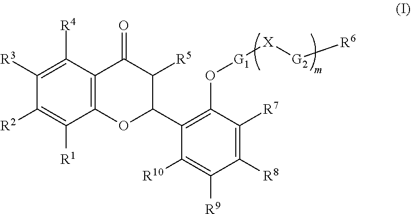
wherein:
R¹ is halogen;
R² is selected from H, OH, halogen, C₁₋₆alkyl, haloC₁₋₆alkyl and C₁₋₆alkoxy;
R³ is selected from H, OH, halogen, C₁₋₆alkyl, haloC₁₋₆alkyl and C₁₋₆alkoxy;
R⁴ is selected from H, OH, halogen, C₁₋₆alkyl, haloC₁₋₆alkyl and C₁₋₆alkoxy;
R⁵ is selected from H, OH, halogen, C₁₋₆alkyl, haloC₁₋₆alkyl and C₁₋₆alkoxy;
R⁶ is selected from OH, carboxy, C₁₋₆alkoxy, C₁₋₆alkoxycarbonyl, carboxycarbonylamino and C₁₋₆ alkoxycarbonylarbonylamino;
R⁷ is selected from H, OH, halogen, C₁₋₆alkyl, haloC₁₋₆alkyl and C₁₋₆alkoxy;
R⁸ is selected from H, OH, halogen, C₁₋₆alkyl, haloC₁₋₆alkyl, C₁₋₆alkoxy and haloC₁₋₆alkoxy;
R⁹ is selected from H, OH, halogen, C₁₋₆alkyl, haloC₁₋₆alkyl and C₁₋₆alkoxy;
or R⁸ and R⁹, together with the atoms to which they are attached, form a heterocyclyl ring;
R¹⁰ is selected from H, OH, halogen, C₁-₆alkyl, haloC₁₋₆alkyl and C₁-₆alkoxy;
G₁ is selected from C₁₋₆alkyl and C₃₋₇cycloalkyl, wherein C₁₋₆alkyl is unsubstituted or substituted by C₁₋₆alkylsulfonylamino, C₃₋₇cycloalkylsulfonylamino, aminosulfonylamino or C₁₋₆ alkylaminosulfonylamino;
X is selected from O and S;
G₂ is selected from C₁₋₆alkyl and C₃₋₇cycloalkyl;
m is 0 or 1;
or a pharmaceutically acceptable salt thereof.

2. A compound according to claim 1, wherein
R² is selected from H, halogen and C₁₋₆alkoxy;
R³ is selected from H, halogen and C₁₋₆alkoxy;
R⁴ is selected from H and OH;
R⁵ is H;
R⁷ is H;
R⁸ is selected from halogen, C₁₋₆alkyl, haloC₁₋₆alkyl, C₁₋₆alkoxy and haloC₁₋₆alkoxy;
R⁹ is selected from H, halogen, C₁₋₆alkyl and C₁₋₆alkoxy;
or R⁸ and R⁹, together with the atoms to which they are attached, form a 5-membered heterocyclyl ring;
R¹⁰ is H;
G₁ is selected from C₁₋₆alkyl and C₃₋₇cycloalkyl; wherein C₁₋₆alkyl is unsubstituted or substituted by C₁₋₆alkylsulfonylamino, aminosulfonylamino or C₁₋₆alkylaminosulfonylamino;
X is O;
or a pharmaceutically acceptable salt thereof.

3. A compound according to claim 2, wherein:
R¹ is Cl;
R² is selected from H, F and methoxy;
R³ is selected from H, F and methoxy;
R⁶ is selected from OH, carboxy, methoxy, methoxycarbonyl, carboxycarbonylamino and ethoxycarbonylcarbonylamino;
R⁸ is selected from Cl, Br, methyl, CF3, methoxy and trifluoromethoxy;
R⁹ is selected from H, Br, methyl and methoxy;
or R⁸ and R⁹, together with the atoms to which they are attached, form a 5-membered heterocyclyl ring;
G₁ is selected from methyl, ethyl, propyl, isobutyl and cyclobutyl; wherein ethyl is unsubstituted or substituted by ethylsulfonylamino, aminosulfonylamino or ethylaminosulfonylamino;
G₂ is selected from methyl and cyclobutyl;
or a pharmaceutically acceptable salt thereof.

4. A compound according to claim 1, or a pharmaceutically acceptable salt thereof, wherein R⁶ is carboxy.

5. A compound according to claim 1, or a pharmaceutically acceptable salt thereof, wherein R⁸ is selected from haloC₁₋₆alkyl and haloC₁₋₆alkoxy.

6. A compound according to claim 5, or a pharmaceutically acceptable salt thereof, wherein R⁸ is selected from CF₃ and trifluoromethoxy.

7. A compound according to claim 1, or a pharmaceutically acceptable salt thereof, wherein R⁹ is selected from H and C₁₋₆alkoxy.

8. A compound according to claim 7, or a pharmaceutically acceptable salt thereof, wherein R⁹ is selected from H and methoxy.

9. A compound according to claim 1, or a pharmaceutically acceptable salt thereof, wherein G₁ is C₁₋₆alkyl.

10. A compound according to claim 9, or a pharmaceutically acceptable salt thereof, wherein G₁ is selected from ethyl and propyl.

11. A compound according to claim 1, or a pharmaceutically acceptable salt thereof, wherein G₂ is C₃₋₇cycloalkyl.

12. A compound according to claim 11, or a pharmaceutically acceptable salt thereof, wherein G₂ is cyclobutyl.

13. A compound according to claim 1, wherein:
R² is selected from H and halogen;
R³ is selected from H and halogen;
R⁴ is H;
R⁵ is H;
R⁷ is H;
R⁶ is —COOH;
R⁸ is selected from haloC₁₋₆alkyl and haloC₁₋₆alkoxy;
R⁹ is selected from H and C₁₋₆alkoxy;
R¹⁰ is H;
G₁ is C₁₋₆alkyl;
G₂ is C₃₋₇cycloalkyl;
X is O;
or a pharmaceutically acceptable salt thereof.

14. A compound according to claim 13, wherein
R¹ is Cl;
R² is selected from H and F;
R³ is selected from H and F;
R⁸ is selected from CF₃ and trifluoromethoxy;
R⁹ is selected from H and methoxy;
G₁ is selected from ethyl and propyl;
G₂ is cyclobutyl;
or a pharmaceutically acceptable salt thereof.

15. A compound according to claim 1, selected from:
3-[2-(8-chloro-4-oxo-chroman-2-yl)-5-(trifluoromethyl)phenoxy]propanoic acid;
3-[2-(8-chloro-7-fluoro-4-oxo-chroman-2-yl)-5-(trifluoromethyl)phenoxy]propanoic acid;
3-[2-(8-chloro-6-fluoro-4-oxo-chroman-2-yl)-5-(trifluoromethyl)phenoxy]propanoic acid;
3-[2-(8-chloro-7-methoxy-4-oxo-chroman-2-yl)-5-(trifluoromethyl)phenoxy]propanoic acid;
3-[2-(8-chloro-6-methoxy-4-oxo-chroman-2-yl)-5-(trifluoromethyl)phenoxy]propanoic acid;
3-[2-(8-chloro-5-hydroxy-4-oxo-chroman-2-yl)-5-(trifluoromethyl)phenoxy]propanoic acid;
3-[5-bromo-2-(8-chloro-4-oxo-chroman-2-yl)-4-methyl-phenoxy]propanoic acid;
3-[5-bromo-2-(8-chloro-4-oxo-chroman-2-yl)-4-methoxy-phenoxy]propanoic acid;
3-[2-(8-chloro-4-oxo-chroman-2-yl)-5-methoxy-4-methyl-phenoxy]propanoic acid;
3-[5-chloro-2-(8-chloro-4-oxo-chroman-2-yl)-4-methoxy-phenoxy]propanoic acid;
3-[2-(8-chloro-4-oxo-chroman-2-yl)-4-methoxy-5-(trifluoromethyl)phenoxy]propanoic acid;
3-[[6-(8-chloro-4-oxo-chroman-2-yl)-1,3-benzodioxol-5-yl]oxy]propanoic acid;
3-[2-(8-chloro-4-oxo-chroman-2-yl)-5-(trifluoromethoxy)phenoxy]propanoic acid;
3-[5-chloro-2-(8-chloro-4-oxo-chroman-2-yl)-4-methyl-phenoxy]propanoic acid;
3-[4-bromo-2-(8-chloro-4-oxo-chroman-2-yl)-5-(trifluoromethoxy)phenoxy]propanoic acid;
3-[5-bromo-2-(8-chloro-4-oxo-chroman-2-yl)phenoxy]propanoic acid;
3-[5-bromo-2-[(2S)-8-chloro-4-oxo-chroman-2-yl]phenoxy]propanoic acid;
3-[5-bromo-2-[(2R)-8-chloro-4-oxo-chroman-2-yl]phenoxy]propanoic acid;
3-[5-chloro-2-(8-chloro-4-oxo-chroman-2-yl)-4-methyl-phenoxy]cyclobutanecarboxylic acid;
3-[2-(8-chloro-4-oxo-chroman-2-yl)-5-(trifluoromethyl)phenoxy]cyclobutanecarboxylic acid;
3-[2-(8-chloro-4-oxo-chroman-2-yl)-5-(trifluoromethyl)phenoxy]-2,2-dimethyl-propanoic acid;
3-[2-[2-(8-chloro-4-oxo-chroman-2-yl)-5-(trifluoromethyl)phenoxy]ethoxy]cyclobutanecarboxylic acid;
2-[2-[2-(8-chloro-4-oxo-chroman-2-yl)-5-(trifluoromethyl)phenoxy]ethoxy]acetic acid;
2-[3-[2-(8-chloro-4-oxo-chroman-2-yl)-5-methyl-phenoxy]propoxy]acetic acid;
2-[3-[2-(8-chloro-4-oxo-chroman-2-yl)-5-(trifluoromethyl)phenoxy]propoxy]acetic acid;
2-[3-[2-(8-chloro-7-fluoro-4-oxo-chroman-2-yl)-5-(trifluoromethyl)phenoxy]propoxy]acetic acid;
4-[2-(8-chloro-7-fluoro-4-oxo-chroman-2-yl)-5-(trifluoromethyl)phenoxy]butanoic acid;
2-[2-(8-chloro-7-fluoro-4-oxo-chroman-2-yl)-5-(trifluoromethyl)phenoxy]acetic acid;
methyl (2R)-3-[2-(8-chloro-4-oxo-chroman-2-yl)-5-(trifluoromethyl)phenoxy]-2-(ethylsulfonylamino)propanoate;
(2R)-3-[2-[(2R)-8-chloro-4-oxo-chroman-2-yl]-5-(trifluoromethyl)phenoxy]-2-(ethylsulfonylamino)propanoic acid;
(2R)-3-[2-[(2S)-8-chloro-4-oxo-chroman-2-yl]-5-(trifluoromethyl)phenoxy]-2-(ethylsulfonylamino)propanoic acid;
(2S)-3-[2-(8-chloro-4-oxo-chroman-2-yl)-5-(trifluoromethyl)phenoxy]-2-(ethylsulfonylamino)propanoic acid;
(2R)-3-[2-[(2R)-8-chloro-4-oxo-chroman-2-yl]-5-(trifluoromethyl)phenoxy]-2-(sulfamoylamino)propanoic acid;
(2R)-3-[2-[(2S)-8-chloro-4-oxo-chroman-2-yl]-5-(trifluoromethyl)phenoxy]-2-(sulfamoylamino)propanoic acid;
(2R)-3-[2-[(2S)-8-chloro-4-oxo-chroman-2-yl]-5-(trifluoromethyl)phenoxy]-2-(ethylsulfamoylamino)propanoic acid;
(2R)-3-[2-[(2R)-8-chloro-4-oxo-chroman-2-yl]-5-(trifluoromethyl)phenoxy]-2-(ethylsulfamoylamino)propanoic acid;
8-chloro-7-fluoro-2-[2-(3-methoxypropoxy)-4-(trifluoromethyl)phenyl]chroman-4-one;
8-chloro-7-fluoro-2-[2-(3-hydroxypropoxy)-4-(trifluoromethyl)phenyl]chroman-4-one;
8-chloro-7-fluoro-2-[2-(2-hydroxyethoxy)-4-(trifluoromethyl)phenyl]chroman-4-one;
ethyl 2-[2-[2-(8-chloro-4-oxo-chroman-2-yl)-5-(trifluoromethyl)phenoxy]ethylamino]-2-oxo-acetate;
2-[2-[5-chloro-2-(8-chloro-4-oxo-chroman-2-yl)phenoxy]ethylamino]-2-oxo-acetic acid; and
cis-3-[5-bromo-2-(8-chloro-4-oxo-chroman-2-yl)phenoxy]cyclobutanecarboxylic acid;
or a pharmaceutically acceptable salt thereof.

16. A compound according to claim 1, selected from:
3-[2-(8-chloro-4-oxo-chroman-2-yl)-5-(trifluoromethyl)phenoxy]propanoic acid;
3-[2-(8-chloro-7-fluoro-4-oxo-chroman-2-yl)-5-(trifluoromethyl)phenoxy]propanoic acid;
3-[2-(8-chloro-6-fluoro-4-oxo-chroman-2-yl)-5-(trifluoromethyl)phenoxy]propanoic acid;
3-[2-(8-chloro-4-oxo-chroman-2-yl)-4-methoxy-5-(trifluoromethyl)phenoxy]propanoic acid;
3-[2-(8-chloro-4-oxo-chroman-2-yl)-5-(trifluoromethoxy)phenoxy]propanoic acid;
3-[2-[2-(8-chloro-4-oxo-chroman-2-yl)-5-(trifluoromethyl)phenoxy]ethoxy]cyclobutane-carboxylic acid; and
4-[2-(8-chloro-7-fluoro-4-oxo-chroman-2-yl)-5-(trifluoromethyl)phenoxy]butanoic acid;
or a pharmaceutically acceptable salt thereof.

17. A process for preparing a compound according to claim 1 comprising at least one of the following steps:
(a) Cyclization of an α,β-unsaturated carbonyl intermediate (XIII), 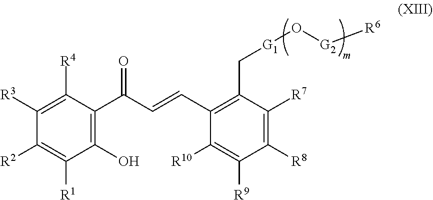
in the presence of a base;
(b) Treatment of a compound of formula (XIX), 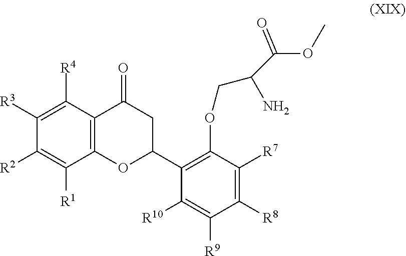
with a compound of formula 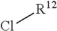
in the presence of a base;
and
(c) Hydrolysis of a compound of formula (I-4), 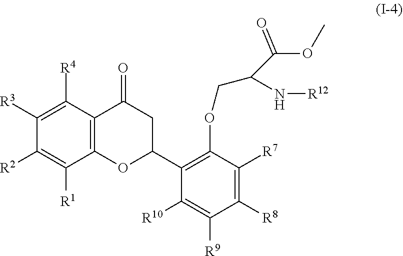
in the presence of a base;
wherein:
R¹ to R¹⁰, G_{1,} G₂ and m are defined as claim 1; and
R¹² is C₁₋₆alkylsulfonyl, aminosulfonyl or C₁₋₆alkylaminosulfonyl.

18. (canceled)

19. A pharmaceutical composition comprising a compound in accordance with claim 1 and a therapeutically inert carrier.

20. (canceled)

21. (canceled)

22. (canceled)

23. (canceled)

24. (canceled)

25. (canceled)

26. (canceled)

27. A compound manufactured according to the process of claim 17.

28. A method for the treatment or prophylaxis of HBV infection, which method comprises administering to a patient in need thereof an effective amount of a compound according to claim 1.

29. A method for the treatment or prophylaxis of HBV infection, which method comprises administering to a patient in need thereof an effective amount of a compound of claim 15.

30. A method of inhibiting a target selected from HBsAg, HBeAg, cccDNA, HBV DNA, the method comprising administering to a patient an effective amount of a compound of claim 1.

31. A method of inhibiting a target selected from HBsAg, HBeAg, cccDNA, HBV DNA, the method comprising administering to a patient an effective amount of a compound of claim 15.

32. A pharmaceutical composition comprising a compound in accordance with claim 15 and a therapeutically inert carrier.


SEQUENCE LISTING (3 sequences)

SEQ ID NO: 1 - DNA, 20 bp, Artificial Sequence
  Description of Artificial Sequence Synthetic primer
aagaaaaaccccgcctgtaa

SEQ ID NO: 2 - DNA, 23 bp, Artificial Sequence
  Description of Artificial Sequence Synthetic primer
cctgttctgactactgcctctcc

SEQ ID NO: 3 - DNA, 27 bp, Artificial Sequence
  Description of Artificial Sequence Synthetic probe
cctgatgtgatgttctccatgttcagc